(19) United States
(12) Patent Application Publication
(10) Pub. No.: US 2022/0194997 A1
(43) Pub. Date: Jun. 23, 2022

(54) SUMO PEPTIDES FOR TREATING NEURODEGENERATIVE DISEASES

(71) Applicant: The Chinese University of Hong Kong, Shatin, N.T. (CN)

(72) Inventors: Michael Kenneth CHAN, Kowloon Tong, Kowloon (CN); Zhaohui LIANG, Shatin, N.T. (CN); Marianne Ming Ming LEE, Kowloon Tong, Kowloon (CN)

(21) Appl. No.: 17/441,922

(22) PCT Filed: Mar. 27, 2020
(86) PCT No.: PCT/CN2020/081616

§ 371 Date: Sep. 22, 2021

Related U.S. Application Data
(60) Provisional application No. 62/825,560, filed on Mar. 28, 2019.

(51) Int. Cl. C07K 14/47; A61K 9/00; C12N 15/63

(52) U.S. Cl. CPC C07K 14/47; A61K 38/00; C12N 15/63; A61K 9/0019

(57) ABSTRACT

Provided are novel compositions, kits, and methods for treating neurodegenerative diseases such as Parkinson's Disease.

### RELATED APPLICATIONS

This application claims priority to U.S. Provisional Patent Application No. 62/825,560, filed Mar. 28, 2019, the contents of which are hereby incorporated by reference in the entirety for all purposes.

### BACKGROUND OF THE INVENTION

Neurodegenerative disease is an umbrella term for a broad range of conditions that primarily affect the neurons in the human brain. Neurons are the building blocks of the nervous system, which includes the brain and spinal cord. Because neurons do not normally reproduce or replace themselves, when they become damaged or die, they cannot be replaced by the body. Neurodegenerative diseases thus can have profound, devastating, yet irreversible effects on those afflicted. The most common neurodegenerative diseases include Parkinson's Disease, Alzheimer's Disease, and Huntington's Disease. Incurable and debilitating, neurodegenerative diseases affect a person's mobility and mental functioning in a progressive and accelerating manner. Worldwide, millions of people are affected by neurodegenerative diseases. By conservative estimates, over 5 million Americans are living with Alzheimer's Disease, and over half a million Americans are living with Parkinson's Disease. Although currently available treatment may help relieve some of the physical or mental symptoms associated with neurodegenerative diseases, there is no known method to slow disease progression or provide cures. As such, cost for caring for individuals suffering from neurodegenerative diseases can be very significant: in 2018, on average it required about $3000 to $6000 per month to maintain a dementia patient.

Because of the prevalence of neurodegenerative diseases, their grave implications on patients' quality of life, and their considerable social economic impact, there exists an urgent need for new and more effective methods for treating neurodegenerative diseases. This invention fulfills this and other related needs.

### BRIEF SUMMARY OF THE INVENTION

The present inventors discovered that Small Ubiquitin-like Modifier (SUMO) proteins and fragments thereof can suppress aggregation of α-synuclein and therefore can suppress the cytotoxicity mediated by α-synuclein.

As such, in the first aspect, the present invention provides a SUMO-derived polypeptide, a nucleic acid, an expression cassette, and related compositions. The SUMO-derived polypeptide comprises a core sequence of the 15-55 or 31-55 segment of SEQ ID NO:1, but does not comprise the full length of SEQ ID NO:1, and the polypeptide is conjugated to a heterologous moiety; and/or the core sequence comprises one or more mutations in the 15-55 or 31-55 segment of SEQ ID NO:1, and the polypeptide suppresses α-synuclein aggregation. For instance, the SUMO-derived polypeptide comprises a core sequence taken from a SUMO protein (thus less than full length of the SUMO protein), optionally modified (e.g., by deletion, insertion, and/or substitution) at one or more amino acid residues, yet retaining at least one of the two β sheets and also the α-helix structures originally present in the wild-type SUMO protein, such that the SUMO-derived polypeptide retains the ability to bind to α-synuclein, for example, via the SIM of α-synuclein. In some embodiments, the core sequence is the 15-55 segment or the 31-55 segment of SEQ ID NO:1, or a corresponding segment in another SUMO protein (e.g., the 10-51 segment of SUMO2, the 10-50 segment of SUMO3, the 10-51 segment of SUMO4, and the 15-55 segment of SUMO5), with optional one or more mutations in the segment (insertion, deletion, and/or substitution). In some embodiments, the SUMO-derived polypeptide comprises a heterologous moiety, and the heterologous moiety is a heterologous amino acid sequence. In some embodiments, the heterologous moiety is a detectable label. In some embodiments, the heterologous moiety is an affinity tag. In some embodiments, the SUMO-derived polypeptide consists of the core sequence and one or more heterologous amino acid sequences at the N- and/or C-terminus of the core sequence. In some embodiments, the SUMO-derived polypeptide consists of the core sequence of the 15-55 or 31-55 segment of SEQ ID NO:1 and a poly-arginine or poly-histidine tag at the N-terminus of the core sequence: for example, the core sequence is the 31-55 segment of SEQ ID NO:1, and a poly-arginine (e.g., 8×Arg) tag or a poly-histidine (e.g., 10×His) tag is present at the N-terminus of the core sequence.

In some embodiments, the present inventions provides a nucleic acid comprising a polynucleotide sequence encoding a SUMO-derived polypeptide described above and herein, e.g., a polypeptide comprising a core sequence of the 15-55 or 31-55 segment of SEQ ID NO:1 (or a corresponding segment in another SUMO protein, e.g., the 10-51 segment of SUMO2, the 10-50 segment of SUMO3, the 10-51 segment of SUMO4, and the 15-55 segment of SUMO5), and the nucleic acid further comprises at least one coding sequence for at least one heterologous amino acid sequence, and/or the core sequence comprises one or more mutations in the 15-55 or 31-55 segment of SEQ ID NO:1 (or a corresponding segment in another SUMO protein, e.g., the 10-51 segment of SUMO2, the 10-50 segment of SUMO3, the 10-51 segment of SUMO4, and the 15-55 segment of SUMO5). In some embodiments, the nucleic acid encodes a fusion protein consisting of the core sequence and one or more heterologous amino acid sequences at the N- and/or C-terminus of the core sequence. In some embodiments, the present invention provides an expression cassette comprising a polynucleotide sequence encoding the SUMO-derived polypeptide described herein and above, which is operably linked to a heterologous promoter. In some embodiments, the polypeptide consists of the core sequence and one or more heterologous amino acid sequences at the N- and/or C-terminus of the core sequence. In some embodiments, the invention provides a vector comprising the expression cassette described above and herein. In some embodiments, a host cell is provided, which comprises the expression cassette or the vector. In some embodiments, a composition is provided that comprises a physiologically acceptable excipient and an effective amount of (1) a SUMO-derived polypeptide comprising the 15-55 or 31-55 segment of SEQ ID NO:1 (or a corresponding segment in another SUMO protein as described herein), optionally comprising one or more mutations in the segment; or (2) a nucleic acid encoding the SUMO-derived polypeptide. In some embodiments, the SUMO-derived polypeptide consists of the 15-55 or 31-55 segment of SEQ ID NO:1. In some embodiments, the SUMO-derived polypeptide consists of the core sequence of the 15-55 or 31-55 segment of SEQ ID NO:1 and a poly-arginine or poly-histidine tag at the N-terminus of the core sequence: for example, the core sequence is the 31-55 segment of SEQ ID NO:1, and a poly-arginine (e.g., 8×Arg) tag or a poly-histidine (e.g., 10×His) tag is present at the N-terminus of the core sequence.

In a second aspect, the present invention provides a method for suppressing α-synuclein aggregation in a cell. The method includes the step of contacting the cell with an effective amount of (1) a SUMO-derived polypeptide comprising the 15-55 or 31-55 segment of SEQ ID NO:1 (or a corresponding segment in another SUMO protein as described herein), optionally comprising one or more mutations in the segment; or (2) a nucleic acid encoding the SUMO-derived polypeptide. In some embodiments, the cell is a neuronal cell. In some embodiments, the neuronal cell is in a human patient's body. In some embodiments, the SUMO-derived polypeptide consists of the 15-55 or 31-55 segment of SEQ ID NO:1. In some embodiments, the SUMO-derived polypeptide consists of the core sequence of the 15-55 or 31-55 segment of SEQ ID NO:1 and a poly-arginine or poly-histidine tag at the N-terminus of the core sequence: for example, the core sequence is the 31-55 segment of SEQ ID NO:1, and a poly-arginine (e.g., 8×Arg) tag or a poly-histidine (e.g., 10×His) tag is present at the N-terminus of the core sequence.

In a third aspect, the present invention provides a method for treating a neurodegenerative disease in a human patient in need thereof. The method comprises administration to the patient an effective amount of: (1) a SUMO-derived polypeptide comprising the 15-55 or 31-55 segment of SEQ ID NO:1 (or a corresponding segment in another SUMO protein as described herein), optionally comprising one or more mutations in the segment; or (2) a nucleic acid encoding the SUMO-derived polypeptide. In some embodiments, the administration comprises intravenous administration or transnasal administration. In some embodiments, the SUMO-derived polypeptide consists of the 15-55 or 31-55 segment of SEQ ID NO:1. In some embodiments, the SUMO-derived polypeptide consists of the core sequence of the 15-55 or 31-55 segment of SEQ ID NO:1 and a poly-arginine or poly-histidine tag at the N-terminus of the core sequence: for example, the core sequence is the 31-55 segment of SEQ ID NO:1, and a poly-arginine (e.g., 8×Arg) tag or a poly-histidine (e.g., 10×His) tag is present at the N-terminus of the core sequence. In some embodiments, the neurodegenerative disease being treated is Parkinson's disease, especially familial Parkinson's disease, as well as Lewy Body Dementia.

In a fourth aspect, the present invention provides a kit for treating a neurodegenerative disease. The kit includes these components; (1) a first container containing a SUMO-derived polypeptide comprising the 15-55 or 31-55 segment of SEQ ID NO:1 (or a corresponding segment in another SUMO protein as described herein), optionally comprising one or more mutations in the segment, or a nucleic acid encoding the SUMO-derived polypeptide; and (2) a second container containing a neuroprotective agent. In some embodiments, the SUMO-derived polypeptide consists of the 15-55 or 31-55 segment of SEQ ID NO:1. In some embodiments, the SUMO-derived polypeptide consists of the core sequence of the 15-55 or 31-55 segment of SEQ ID NO:1 and a poly-arginine or poly-histidine tag at the N-terminus of the core sequence: for example, the core sequence is the 31-55 segment of SEQ ID NO:1, and a poly-arginine (e.g., 8×Arg) tag or a poly-histidine (e.g., 10×His) tag is present at the N-terminus of the core sequence. In some embodiments, the neurodegenerative disease being treated is Parkinson's disease, especially familial Parkinson's disease, as well as Lewy Body Dementia. In some embodiments, the kit further includes an instruction manual providing instructions for the user to use the contents of the kit for its intended purposes.

### BRIEF DESCRIPTION OF THE DRAWINGS

FIG. 1. The dose-dependent effect of SUMO1(15-55) on inhibition of α-synuclein aggregation. 70 μM α-synuclein was mixed with SUMO1(15-55) in different ratios (α-synuclein: SUMO1(15-55)=1:1, 1:0.5, 1:0.2, 1:0.1) and incubated at 37° C. for consecutive 7 days. (A) Effect of different ratios of SUMO1(15-55) on the aggregation of α-synuclein. (The line overlapping with the abscissa axis: buffer only) (B) Size-exclusion chromatography analysis of protein samples from fibrillization assay. At the end of fibrillizaton assay protein, samples were subjected to a Superdex 200 GL 5/150 column. (C) The time course of amyloidogenesis of protein samples in (I-M) respectively. (D-H) CD spectra of α-synuclein alone (D) and mixtures of α-synuclein/SUMO1(15-55) at ratios of (E) 1:1, (F) 1:0.5, (G)1:0.2, (H) 1:0.1 were recorded at the indicated incubation times. (I-M) TEM images of the α-synuclein/SUMO1(15-55) mixtures from fibrillization assay after 7 days incubation for (I) α-synuclein alone, and at ratios of α-synuclein/SUMO1(15-55) of (J) 1:1, (K) 1:0.5, (L) 1:0.2, (M) 1:0.1. Scar bar: 500 nm.

FIG. 2. Neuroprotective effect of SUMO1.(1.5-55) on SH-SY5Y cells, (A) The toxicity of α-synuclein aged in the absence and presence of stoichiometric amounts of SUMO1(15-55) on SH-SY5Y cells. (B) The toxicity of NACore aged in the absence and presence of stoichiometric amounts of SUMO1(15-55) on SH-SY5Y cells. (C) Confocal images showing cellular uptake of α-synuclein aged in the absence and presence of stoichiometric amounts of SUMO1(15-55), (D-E) Quantitation of cellular uptake by flow cytometry. Data analysis was performed using one-way ANOVA followed by Dunnett's post hoc test; values represent mean±SD (**p<0.01, ****p <0.0001, compared with α-synuclein/NACore only).

FIG. 3. Visualization and quantification of protein interactions by coupling biomolecular fluorescence complementation and flow cytometry. (A) SH-SY5Y cells transfected with corresponding plasmids, and BiFC signals were detected by confocal microscope. Positive control: mVenus. Negative control: VN173+SUMO1(15-55)-VC155. Scar bar: 25 μm. (B) Representative BiFC-FC scatter plots of individual samples. Percentages refer to BiFC-positive cells. (C) Quantitation of BiFC-positive SH-SY5Y cells co-transfected with corresponding plasmids. (D) Quantitation of the mean fluorescence value of BiFC-positive Data analysis was performed using one-way ANOVA followed by Dunnett's post hoc test; values represent mean±SD (**p<0.01, ****p<0.0001, compared with negative control.

FIG. 4. SUMO1(15-55) suppresses photoreceptor neurodegeneration. Larval feeding of SUMO1(15-55) suppressed photoreceptor degeneration in α-synuclein transgenic Drosophila quatitated 3-4 days post eclosion. (A) Representative images of visible rhabdomeres in Drosophila eyes. (B) The average number of rhabdomeres per ommatidium. (C) The distribution of the percent of ommatidia. For each condition, at least 650 ommatidia from 35-40 flies obtained from three independent crosses were used to calculate the average number of rhabdomeres per ommatidium. Data analysis was performed using one-way ANOVA followed by Dunnett's post hoc test; values represent mean±SEM (****p<0.0001, compared with control group).

FIG. 5. SUMO1(15-55) improved locomotor dysfunction by protecting dopaminergic neurons. Climbing ability of adult α-synuclein transgenic Drosophila aged day 1, 4, 7, 10 and 13 post eclosion. (A) Locomotor dysfunction of α-synuclein transgenic Drosophila was rescued by larval feeding of SUMO1(15-55) in a dose-dependent manner. (B) Representative Western blots detecting protein expression levels in the heads of 13 day aged α-synuclein transgenic Drosophila. (C) Quantitation of TH expression level. (D) Quantitation of α-synuclein expression level. Data analysis was performed using one-way ANOVA followed by Dunnett's post hoc test; values represent mean±SEM (*p<0.05, ***p<0.001, ****p<0.0001 compared with no treatment group or control group).

FIG. 6. Inhibition of SDS-induced alpha-synuclein aggregation by SUMO1 derived peptides at 1:1 concentration. Fluorescence intensity as measured by ThT assay. These data show that SUMO1(31-55) has similar ability to SUMO1(15-55) at inhibiting alpha-synuclein aggregation. However, SUMO1(20-40) is not effective in inhibiting alpha-synuclein aggregation. These data indicate that the sheet-loop-helix motif of SUMO1(31-55) is sufficient for inhibiting alpha-synuclein aggregation by SUMO1 derived peptides.

FIG. 7. Determination of the binding of SUMO(31-55) to alpha-synuclein by microscale thermophoresis (MST). These data indicate that SUMO1(31-55) has similar affinity as SUMO1(15-55) in binding to alpha-synuclein.

FIG. 8. Sequence alignment of SUMO1-5 proteins. A. Reproduced from Liang, Y. C. et al. “SUMO5, a Novel Poly-SUMO Isoform, Regulates PML Nuclear Bodies”, Sci Rep. 2016 May 23; 6:26509. doi: 10.1038/srep26509. B. SUMO1 (15-55) and corresponding segments in other SUMO proteins.

FIG. 9. Structure of SUMO1 protein. (A) SUMO1(15-55) (dark grey). (B) SUMO1(31-55) (white). (C) SUMO1(20-40) (dark grey). The remaining residues of the SUMO1 protein are indicated in light grey.

FIG. 10. Insight into the site on SUMO1(15-55) peptide that binds to α-synuclein. a. Substitution of two hydrophobic residues into hydrophilic residues diminishes the hydrophobicity of the putative binding groove. Surface representation of SUMO1(15-55) (left) and SUMO1(15-55) (L44E, L47R) (right). And close-up of the regions highlighted in black box. Hydrophilic residues (dark grey), hydrophobic residues (light grey). The salt bridge is shown with purple dotted line. b. Predicted model structures of the SUMO1(15-55)-αsyn(35-45) complex (left) and SUMO1(15-55) (L44E, L47R)-αsyn(35-45) complex (right) indicate that αsyn(35-45) fails to bind to the mutant. All predicted complexes structures were obtain by docking αsyn(35-45) (black) to SUMO1(15-55) constructs (grey) (derived from PDB ID: 2N1V) using PIPER-FlexDock. c. The aggregation kinetic of α-synuclein in the absence and presence of SUMO1(15-55)/SUMO1(15-55)(L44E, L47R) in equimolar ratio. Buffer only (The line overlapping with the abscissa axis). Data are presented as mean±s.e.m. (n=4). d. MST assays for the binding of SUMO1(15-55)/SUMO1(L44E, L47R) and α-synuclein to investigate the effect of hydrophobicity in the interaction. Error bars represent mean±s.e.m. based on at least three independent measurements. Binding curves and Kd values are shown.

FIG. 11. Insight into the SUMO1(15-55) binding site on α-synuclein. a. The amino acid sequence of full-length human wild-type α-synuclein protein. Residues 35-45 are coloured in magenta, residues 48-60 are coloured in green. SIM/SIM-like sequences are underlined. b. MST assays for the binding of SUMO1(15-55) and α-synuclein fragment peptides to narrow down the interaction regions on α-synuclein. c. MST assays for the binding of SUMO1(15-55) and quintuple alanine (5A) mutants of α-synuclein SIM1 and SIM2 to investigate the sequence selection in the interaction. Error bars represent mean±s.e.m. based on at least three independent measurements. Binding curves and Kd values are shown. d. Predicted model structure of the SUMO1(15-55)-αsyn(35-45) complex obtained by docking αsyn(35-45) (magenta) to SUMO1(15-55) (grey) (derived from PDB ID: 2N1V) using PIPER-FlexPepDock protocol. The close-up and clipped view show that αsyn(35-45) binds to the putative hydrophobic binding groove via SIM (³⁷VLYV⁴⁰) as expected. Y39 is shown in yellow, the hydrophobic residues are shown in orange.

FIG. 12. SUMO1(15-55) suppresses neurodegeneration in α-synuclein transgenic Drosophila. Photoreceptor neurodegeneration was suppressed by larval feeding of SUMO1(15-55) in a dose-dependent manner in α-synuclein-A30P transgenic Drosophila a or α-synuclein-A53T transgenic Drosophila c. Corresponding frequency distributions are shown in b, d. Representative images of visible rhabdomeres in Drosophila eyes. For each condition, at least 100 ommatidia collected from 10 fly eyes were examined. The mean number of photoreceptors s.e.m. per ommatidium is indicated at the bottom of each panel.

FIG. 13. SUMO1(31-55) ameliorates the locomotor dysfunction in α-synuclein transgenic Drosophila. Locomotor dysfunction was rescued by larval feeding of SUMO1(15-55) in a dose-dependent manner (n=20 drosophila per group) in α-synuclein transgenic Drosophila a. α-synuclein-A30P transgenic Drosophila b. α-synuclein-A53T transgenic Drosophila c. Data represent mean±s.e.m. P values were calculated by two-way ANOVA followed by Dunnett's post-hoc test. *p<0.05, **p<0.01, ***p<0.001, ****p<0.0001 vs. the no treatment control. ns, not significant.

FIG. 14. Internalization of R₈-SUMO1(31-55) in SH-SY5Y cells and its inhibitory effect in vitro. a. Representative images of cellular uptake of R8-SUMO1(31-55) in a time-course manner. R₈-SUMO1(31-55) was labelled with NHS-Alexa 488 before co-incubation with SH-SY5Y cells. Cells were fixed at different time points (1 h and 24 h) and the nuclei were stained with Hoechst 33342 (grey spots), cell membrane was stained with wheat germ agglutinin (grey spots). b. The aggregation kinetic of α-synuclein in the absence and presence of R₈-SUMO1(31-55) in stoichiometric amounts. Buffer only (The line overlapping with the abscissa axis). Data are presented as mean±s.e.m. (n=4).

FIG. 15. Inhibition effect of His-SUMO1(31-55) in vitro. The aggregation kinetic of α-synuclein in the absence and presence of His-SUMO1(31-55) in stoichiometric amounts. Buffer only (The line overlapping with the abscissa axis). Data are presented as mean±s.e.m. (n=4).

FIG. 16. Validation of the SUMO1(15-55) binding site on α-synuclein. a, b Predicted model structures of the SUMO1(15-55)-αsyn(35-45) complex a and SUMO1(15-55)-αsyn(48-60) complex b obtained by docking αsyn(35-45) (medium grey as shown in the enlarged area of a) or αsyn(48-60) (medium grey as shown in the enlarged area of b) to SUMO1(15-55) using PIPER-FlexPepDock protocol. The close-up and clipped views show that αsyn(35-45)/αsyn(48-60) binds to the putative hydrophobic binding groove via SIM1 (³⁷VLYV⁴⁰)/SIM2 (⁴⁸VVHGV⁵²) as expected. The hydrophobic residues are shown in orange. c. MST assays for the binding of SUMO1(15-55) and α-synuclein mutants. Error bars represent mean±s.e.m. based on at least three independent measurements. Binding curves and Kd values are shown.

FIG. 17. Effect of different ratios of SUMO2(16-88) (identical sequence to SUMO3(15-87)) on the aggregation of α-synuclein. 70 μM α-synuclein was mixed with SUMO1(16-88) in different ratios (α-synuclein: SUMO1(16-88)=1:1, 1:0.5, 1:0.2, 1:0.1) and incubated at 37° C. for consecutive 7 days.

FIG. 18. Effect of different ratios of SUMO2(16-51) (identical sequence to SUMO3(15-50)) on the aggregation of α-synuclein. 70 μM α-synuclein was mixed with SUMO1(16-51) in different ratios (α-synuclein: SUMO1(16-51)=1:1, 1:0.5, 1:0.2, 1:0.1) and incubated at 37° C. for consecutive 7 days.

### DEFINITIONS

The term “SUMO” or “Small Ubiquitin-like Modifier,” as used herein, refers to a family of small proteins that modify other proteins' functions within cells by covalently attaching to and detaching from such proteins. Most SUMO proteins are small: about 100 amino acids in length and 12 kDa in molecular weight. Similar to ubiquitin, SUMO proteins are considered members of the ubiquitin-like protein family. Dissimilar to ubiquitin, SUMO proteins do not tag proteins for degradation, although SUMOylation is directed by an enzymatic cascade analogous to that involved in ubiquitination. A post-translational modification process, SUMOylation is involved in various cellular processes, such as nuclear-cytosolic transport, transcriptional regulation, apoptosis, protein stability, response to stress, and progression through the cell cycle. SUMO proteins have been found in many species. There are 4 confirmed SUMO isoforms in humans: SUMO-1, SUMO-2, SUMO-3, and SUMO-4. In addition, there is the newly discovered SUMO5, a novel primate-specific and tissue-specific small ubiquitin-like modifier protein. Human SUMO1 protein has the amino acid sequence set forth in SEQ ID NO:1 and GenBank Accession No. AAC50996.1, and its coding sequence set forth in SEQ ID NO:2 and GenBank Accession No. NG 011679.1. Human SUMO1 is a globular protein with a spherical core consisting of an alpha helix and a beta sheet, both ends of the polypeptide chain protruding from the protein's core.

As used herein a “SUMO-derived polypeptide” refers to a polypeptide comprising a core sequence that generally corresponds to a fragment (such as the 15-55 fragment or the 31-55 fragment) of a SUMO protein (especially the human SUMO1 protein) and retains the ability to inhibit α-synuclein aggregation (e.g., as determined in a fibrillation/Thioflavin T (ThT) assay, an α-synuclein cytotoxicity assay in neuronal cells, or a neuroprotective assay in a Parkinson's disease model such as α-synuclein transgenic Drosophila). For instance, the core sequence may include at least 30, 31, 32, 33, 34, 35, 36, 37, 38, 39, 40 or up to 41 amino acid residues identical to their corresponding residues in the 15-55 segment of human SUMO1 protein. Alternatively, the core sequence may include at least 15, 16, 17, 18, 19, 20, 21, 22, 23, 24 or up to 25 amino acid residues identical to segment 31-55 of the human SUMO1 protein. In other words, possible changes or mutations (e.g., deletion, insertion, or substitution, especially conservative substitution) within these core sequences typically involve no more than 1, 2, 3, 4, 5, 6, 7, 8, 9, 10, or 11 residues, with the cysteine residue at position 52 optionally preserved. A “SUMO1-derived polypeptide” in some cases may encompass a full length SUMO protein while in other cases does not encompass the full length SUMO protein sequence. It can include one or more heterologous polypeptide sequences at the N- and/or C-terminus of the core sequence corresponding to a SUMO protein fragment. A “SUMO-derived polypeptide” may contain, in the core sequence and/or in the heterologous sequence(s), one or more modified or artificial amino acids, such as D-amino acids, as well as modifications such as glycosylation or PEGylation. In addition, other human SUMO proteins are known (the amino acid sequence for human SUMO2 protein is set forth in GenBank Accession No. AAH71645.1; the amino acid sequence for human SUMO3 protein is set forth in NCBI Reference Sequence: NP 008867.2; the amino acid sequence for human SUMO4 protein is set forth in NCBI Reference Sequence NP 001002255.1; and human SUMO5 gene is set forth in GenBank Accession No. FJ042790.1, its protein amino acid sequence set forth in SEQ ID NO:6), as well as fragments derived from these proteins comprising the segment corresponding to the 31-55 segment of SEQ ID NO:1 (human SUMO1 protein amino acid sequence) are also expected to possess the same or similar activity in binding to α-synuclein and inhibiting α-synuclein aggregation. Sequence alignment indicates SUMO1, SUMO2, SUMO3, SUMO4, SUMO5 share similar amino acid sequences. SUMO2, SUMO3, and SUMO4 have similar β1, β2-strands and α-helix. The fragments corresponding to SUMO1(15-55) are SUMO2(10-51), SUMO3(10-50), SUMO4(10-51), and SUMO5(15-55). The fragments corresponding to SUMO1(31-55) are SUMO2(27-51), SUMO3(26-50), SUMO4(27-51), and SUMO5(31-55). The fragments corresponding to SUMO2(16-88) are SUMO3(15-87), SUMO1(20-92), SUMO4(16-88), and SUMO5(20-92). The fragments corresponding to SUMO2(16-51) are SUMO3(15-50), SUMO1(20-55), SUMO4(16-51), and SUMO5(20-55).

In this disclosure, the term “neurodegenerative disease” or “neurodegenerative disorder” includes, but is not limited to the following conditions: diseases of central motor systems including degenerative conditions affecting the basal ganglia (Huntington's disease, Wilson's disease, striatonigral degeneration, corticobasal ganglionic degeneration), Tourette's syndrome, Parkinson's disease (PD, especially familial PD), progressive supranuclear palsy, progressive bulbar palsy, familial spastic paraplegia, spinomuscular atrophy, amyotrophic lateral sclerosis (ALS) and variants thereof, dentatorubral atrophy, olivo-pontocerebellar atrophy, paraneoplastic cerebellar degeneration, and dopamine toxicity; diseases affecting sensory neurons such as Friedreich's ataxia, diabetes, peripheral neuropathy, and retinal neuronal degeneration; diseases of limbic and cortical systems such as cerebral amyloidosis, Pick's atrophy, and Retts syndrome; neurodegenerative pathologies involving multiple neuronal systems and/or brainstem including Alzheimer's disease, Parkinson's disease, AIDS-related dementia, Leigh's disease, diffuse Lewy body disease, epilepsy, multiple system atrophy, Guillain-Barre syndrome, lysosomal storage disorders such as lipofuscinosis, late-degenerative stages of Down's syndrome, Alper's disease, vertigo as result of central nervous system (CNS) degeneration, ALS, corticobasal degeneration, and progressive supranuclear palsy; pathologies associated with developmental retardation and learning impairments, Down's syndrome, and oxidative stress induced neuronal death; pathologies arising with aging and chronic alcohol or drug abuse including, for example, (i) with alcoholism, the degeneration of neurons in locus coeruleus, cerebellum, cholinergic basal forebrain, (ii) with aging, degeneration of cerebellar neurons and cortical neurons leading to cognitive and motor impairments, and (iii) with chronic amphetamine abuse, degeneration of basal ganglia neurons leading to motor impairments; pathological changes resulting from focal trauma such as stroke, focal ischemia, vascular insufficiency, hypoxic-ischemic encephalopathy, hyperglycemia, hypoglycemia, closed head trauma, and direct trauma; pathologies arising as a negative side-effect of therapeutic drugs and treatments (e.g., degeneration of cingulate and entorhinal cortex neurons in response to anticonvulsant doses of antagonists of the NMDA class of glutamate receptor). In particular, “neurodegenerative disease” or “neurodegenerative disorder” is used to refer to a neurological disease or disorder in which Lewy bodies are present, for example, Parkinson's Disease, Lewy body dementia (LBD, also known as Lewy body disorder, which includes Parkinson's disease dementia (PDD) and dementia with Lewy bodies (DLB), two types of dementia characterized by abnormal deposits of α-synuclein protein in the brain), and multiple system atrophy (MSA), also known as Shy-Drager syndrome.

In this disclosure the term “or” is generally employed in its sense including “and/or” unless the content clearly dictates otherwise.

In this disclosure the term “isolated” nucleic acid molecule means a nucleic acid molecule that is separated from other nucleic acid molecules that are usually associated with the isolated nucleic acid molecule. Thus, an “isolated” nucleic acid molecule includes, without limitation, a nucleic acid molecule that is free of nucleotide sequences that naturally flank one or both ends of the nucleic acid in the genome of the organism from which the isolated nucleic acid is derived (e.g., a cDNA or genomic DNA fragment produced by PCR or restriction endonuclease digestion). Such an isolated nucleic acid molecule is generally introduced into a vector (e.g., a cloning vector or an expression vector) for convenience of manipulation or to generate a fusion nucleic acid molecule. In addition, an isolated nucleic acid molecule can include an engineered nucleic acid molecule such as a recombinant or a synthetic nucleic acid molecule. A nucleic acid molecule existing among hundreds to millions of other nucleic acid molecules within, for example, a nucleic acid library (e.g., a cDNA or genomic library) or a gel (e.g., agarose, or polyacrylamine) containing restriction-digested genomic DNA, is not an “isolated” nucleic acid.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid. For example, both D- and L-amino acids are within the scope of “amino acids” in this disclosure.

There are various known methods in the art that permit the incorporation of an unnatural amino acid derivative or analog into a polypeptide chain in a site-specific manner, see, e.g., WO 02/086075.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The term “recombinant” when used with reference, e.g., to a cell, or a nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

A “promoter” is defined as an array of nucleic acid control sequences that direct transcription of a polynucleotide sequence. As used herein, a promoter includes necessary polynucleotide sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a polynucleotide expression control sequence (such as a promoter, or array of transcription factor binding sites) and a second polynucleotide sequence, wherein the expression control sequence directs transcription of the polynucleotide sequence corresponding to the second sequence.

An “expression cassette” is a nucleic acid construct, generated recombinantly or synthetically, with a series of specified polynucleotide elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter.

The term “heterologous” as used in the context of describing the relative location of two elements, refers to the two elements such as polynucleotide sequences (e.g., a promoter or a protein/polypeptide-encoding sequence) or polypeptide sequences (e.g., two peptides as fusion partners within a fusion protein) that are not naturally found in the same relative positions. Thus, a “heterologous promoter” of a gene refers to a promoter that is not naturally operably linked to that gene. Similarly, a “heterologous polypeptide” or “heterologous polynucleotide” to a particular protein or its encoding sequence is one derived from an origin that is different from that particular protein, or if derived from the same origin but not naturally connected to that particular protein or its coding sequence in the same fashion. The fusion of one polypeptide (or its coding sequence) with a heterologous polypeptide (or polynucleotide sequence) does not result in a longer polypeptide or polynucleotide sequence that can be found in nature. A “heterologous” fusion partner to a SUMO-derived peptide (e.g., the 15-55 or 31-55 segment of SEQ ID NO:1) is another peptide from a non-SUMO origin, for example, a poly-His tag (e.g., a tag comprising 6, 8, or 10 or more His) for ease of purification or a poly-Arg tag (e.g., a tag comprising 6, 8, 10, or more Arg) for intracellular translocation.

A “label,” “detectable label,” or “detectable moiety” is a composition detectable by radiological, spectroscopic, photochemical, biochemical, immunochemical, chemical, or other physical means. For example, useful labels include radioisotopes such as ³²P, fluorescent dyes, electron-dense reagents, enzymes (e.g., as commonly used in an ELISA), biotin, digoxigenin, or haptens and proteins that can be made detectable, e.g., by incorporating a radioactive component into a polypeptide or used to detect antibodies specifically reactive with the polypeptide. Typically a detectable label is a heterologous moiety attached to a probe or a molecule (e.g., a protein or nucleic acid) with defined binding characteristics (e.g., a polypeptide with a known binding specificity or a polynucleotide), so as to allow the presence of the probe/molecule (and therefore its binding target) to be readily detectable. The heterologous nature of the label ensures that it has an origin different from that of the probe or molecule that it labels, such that the probe/molecule attached with the detectable label does not constitute a naturally occurring composition (e.g., a naturally occurring polynucleotide or polypeptide sequence).

By “host cell” is meant a cell that contains an expression vector and supports the replication or expression of the expression vector. Host cells may be prokaryotic cells such as E. coli, or eukaryotic cells such as yeast, insect, amphibian, or mammalian cells such as CHO, HeLa and the like, e.g., cultured cells, explants, and cells in vivo.

The term “inhibiting” or “inhibition,” as used herein, refers to any detectable negative effect on a target biological process, such as protein phosphorylation, cellular signal transduction, protein synthesis, cell proliferation, tumorigenicity, and metastatic potential etc. Typically, an inhibition is reflected in a decrease of at least 10%, 20%, 30%, 40%, or 50% in target process (e.g., α-synuclein aggregation or cytotoxicity mediated by α-synuclein), or any one of the downstream parameters mentioned (e.g., neuronal cell death due to α-synuclein toxicity), when compared to a control. In a similar fashion, the term “increasing” or “increase” is used to describe any detectable positive effect on a target biological process, for example, an effect of neuroprotection for neuronal cells against α-synuclein cytotoxicity, such as a positive change of at least 25%, 50%, 75%, 100%, or as high as 2, 3, 4, 5 or up to 10 or 20 fold, when compared to a control.

The term “effective amount,” as used herein, refers to an amount that is sufficient to produces an intended effect for which a substance is administered. The effect may include a desirable change in a biological process (e.g., decreased α-synuclein aggregation or α-synuclein cytotoxicity) as well as the prevention, correction, or inhibition of progression of the symptoms of a disease/condition and related complications to any detectable extent. The exact amount “effective” for achieving a desired effect will depend on the nature of the therapeutic agent, the manner of administration, and the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

The term “about” denotes a range of +/−10% of a pre-determined value. For example, “about 10” sets a range of 90% to 110% of 10, i.e., 9 to 11.

### DETAILED DESCRIPTION OF THE INVENTION

### I. INTRODUCTION

The misfolding and aggregation of protein α-synuclein results in the formation of amyloid fibrils in brain, and in turn synucleinopathies that include the second most common neurodegenerative disease—Parkinson's disease (PD). Previous studies have shown that sumoylation of α-synuclein occurs naturally in the brain, and this sumoylated α-synuclein can suppress α-synuclein aggregation. This study reports that the variants of small ubiquitin-like modifier 1 (SUMO1) protein can directly suppress α-synuclein aggregation in vitro with an efficiency that appears comparable to that reported for sumoylated-α-synuclein.

After systematically preparing and testing a series of SUMO1 variants, the present inventors developed a peptide as a potential therapeutic lead for in vivo studies. This peptide is based on a core region of SUMO1 (residues 15-55) containing two beta sheets and an alpha-helix, or based on a core region of an even shorter, further truncated SUMO segment (residues 31-55) consisting of a beta sheet followed by an alpha-helix, both core regions containing binding residues involved in SUMO-SUMO interaction motif (SIM) interactions. The SUMO1(15-55) and (31-55) peptides exhibited improved suppression activity relative to SUMO1 itself in in vitro studies. Also, the SUMO1(15-55) peptide reduced cytotoxicity and blocked aggregated α-synuclein transmission between SH-SY5Y cells. Larval feeding of SUMO1(15-55) peptide significantly ameliorated the disease symptoms in a Drosophila PD model by suppressing the loss of dopaminergic neurons. To elucidate the interaction region of SUMO(15-55) binding to α-synuclein, crosslinking studies and microscale thermophoresis were employed to test the predicted regions. Data suggest that binding of SUMO(15-55) is localized near the predicted SIM motif in α-synuclein (residues 37-41) and a hydrophobic stretch in α-synuclein (residues 74-84) known to be important in suppression of α-synuclein aggregation. These findings show that SUMO1 has a direct role in suppressing α-synuclein aggregation and provide an avenue for developing new treatments for PD and/or other synucleinopathies.

The study disclosed herein thus provides a SUMO-derived peptide, which comprises a core sequence taken from a SUMO protein (hence less than full length of the SUMO protein), optionally modified (e.g., deletion, insertion, or substitution) at one or more amino acid residues, yet retaining at least one of the two beta sheets and also the alpha-helix structures, such that the SUMO-derived peptide retains the ability to bind to α-synuclein, for example, via the SIM of α-synuclein. This binding capability is readily verifiable by in vitro binding assays or cross-linking assays known in the art or described herein using a polypeptide comprising at least a fragment of the α-synuclein protein including the SIM.

### II. RECOMBINANT EXPRESSION OF POLYPEPTIDES

### A. General Recombinant Technology

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, J. Chrom. 255: 137-149 (1983).

The polynucleotide sequence encoding a polypeptide of interest, e.g., a SUMO-derived polypeptide, and synthetic oligonucleotides can be verified after cloning or subcloning using, e.g., the chain termination method for sequencing double-stranded templates of Wallace et al., Gene 16: 21-26 (1981).

### B. Cloning and Subcloning of a Coding Sequence

The polynucleotide sequences encoding human SUMO1 is known as GenBank Accession No. NG 011679.1 and set forth in SEQ ID NO:2. The corresponding amino acid sequence is known as GenBank Accession No. AAC50996.1 and set forth in SEQ ID NO: 1. These polynucleotide sequences may be obtained from a commercial supplier or by amplification methods such as polymerase chain reaction (PCR).

The rapid progress in the studies of human genome has made possible a cloning approach where a human DNA sequence database can be searched for any gene segment that has a certain percentage of sequence homology to a known nucleotide sequence. Any DNA sequence so identified can be subsequently obtained by chemical synthesis and/or PCR technique such as overlap extension method. For a short sequence, completely de novo synthesis may be sufficient; whereas further isolation of full length coding sequence from a human cDNA or genomic library using a synthetic probe may be necessary to obtain a larger gene.

Alternatively, a polynucleotide sequence encoding a SUMO polypeptide can be isolated from a cDNA or genomic DNA library using standard cloning techniques such as PCR, where homology-based primers can often be derived from a known nucleic acid sequence encoding a SUMO polypeptide. This approach is particularly useful for identifying variants, orthologs, or homologs of any particular SUMO protein. Most commonly used techniques for this purpose are described in standard texts, e.g., Sambrook and Russell, supra.

cDNA libraries suitable for obtaining a coding sequence for a human SUMO, especially SUMO1, polypeptide may be commercially available or can be constructed. The general methods of isolating mRNA, making cDNA by reverse transcription, ligating cDNA into a recombinant vector, transfecting into a recombinant host for propagation, screening, and cloning are well known (see, e.g., Gubler and Hoffman, Gene, 25: 263-269 (1983); Ausubel et al., supra). Upon obtaining an amplified segment of nucleotide sequence by PCR, the segment can be further used as a probe to isolate the full length polynucleotide sequence encoding the gene of interest (e.g., human SUMO1) from the cDNA library. A general description of appropriate procedures can be found in Sambrook and Russell, supra. A similar procedure can be followed to obtain a sequence encoding a human SUMO1 from a human genomic library, which may be commercially available or can be constructed according to various art-recognized methods. Based on sequence homology, degenerate oligonucleotides can be designed as primer sets and PCR can be performed under suitable conditions (see, e.g., White et al., PCR Protocols: Current Methods and Applications, 1993; Griffin and Griffin, PCR Technology, CRC Press Inc. 1994) to amplify a segment of nucleotide sequence from a cDNA or genomic library.

Upon acquiring a polynucleotide sequence encoding a human SUMO1 sequence, the sequence can be modified and then subcloned into a vector, for instance, an expression vector, so that a recombinant polypeptide (e.g., a SUMO-derived polypeptide) can be produced from the resulting construct. Further modifications to the coding sequence, e.g., nucleotide substitutions, may be subsequently made to alter the characteristics of the polypeptide.

### C. Modification of a Polynucleotide Coding Sequence

The amino acid sequence of a SUMO protein or fragment may be modified in order to achieve the desired functionality of inhibiting α-synuclein aggregation, as determined by the in vitro or in vivo methods known in the field as well as described herein, e.g., the Thioflavin T (ThT) assay, α-synuclein cytotoxicity assay in neuroblastoma cells, or neuroprotective assay in α-syneuclein transgenic Drosophila. Possible modifications to the amino acid sequence may include conservative substitutions; deletion or addition of one or more amino acid residues (e.g., addition at one terminal of the polypeptide of a tag sequence such as 10×His to facilitate purification or identification) at either or both of the N- and C-termini.

A variety of mutation-generating protocols are established and described in the art, and can be readily used to modify a polynucleotide sequence encoding a SUMO-derived polypeptide. See, e.g., Zhang et al., Proc. Natl. Acad. Sci. USA, 94: 4504-4509 (1997); and Stemmer, Nature, 370: 389-391 (1994). The procedures can be used separately or in combination to produce variants of a set of nucleic acids, and hence variants of encoded polypeptides. Kits for mutagenesis, library construction, and other diversity-generating methods are commercially available.

Mutational methods of generating diversity include, for example, site-directed mutagenesis (Botstein and Shortle, Science, 229: 1193-1201 (1985)), mutagenesis using uracil-containing templates (Kunkel, Proc. Natl. Acad. Sci. USA, 82: 488-492 (1985)), oligonucleotide-directed mutagenesis (Zoller and Smith, Nucl. Acids Res., 10: 6487-6500 (1982)), phosphorothioate-modified DNA mutagenesis (Taylor et al., Nucl. Acids Res., 13: 8749-8764 and 8765-8787 (1985)), and mutagenesis using gapped duplex DNA (Kramer et al., Nucl. Acids Res., 12: 9441-9456 (1984)).

Other possible methods for generating mutations include point mismatch repair (Kramer et al., Cell, 38: 879-887 (1984)), mutagenesis using repair-deficient host strains (Carter et al., Nucl. Acids Res., 13: 4431-4443 (1985)), deletion mutagenesis (Eghtedarzadeh and Henikoff, Nucl. Acids Res., 14: 5115 (1986)), restriction-selection and restriction-purification (Wells et al., Phil. Trans. R. Soc. Lond. A, 317: 415-423 (1986)), mutagenesis by total gene synthesis (Nambiar et al., Science, 223: 1299-1301 (1984)), double-strand break repair (Mandecki, Proc. Natl. Acad. Sci. USA, 83: 7177-7181 (1986)), mutagenesis by polynucleotide chain termination methods (U.S. Pat. No. 5,965,408), and error-prone PCR (Leung et al., Biotechniques, 1: 11-15 (1989)).

### D. Modification of Nucleic Acids for Preferred Codon Usage in a Host Organism

The polynucleotide sequence encoding a SUMO-derived polypeptide can be further altered to coincide with the preferred codon usage of a particular host. For example, the preferred codon usage of one strain of bacterial cells can be used to derive a polynucleotide that encodes a SUMO-derived polypeptide includes the codons favored by this strain. The frequency of preferred codon usage exhibited by a host cell can be calculated by averaging frequency of preferred codon usage in a large number of genes expressed by the host cell (e.g., calculation service is available from web site of the Kazusa DNA Research Institute, Japan). This analysis is preferably limited to genes that are highly expressed by the host cell.

At the completion of modification, the coding sequences are verified by sequencing and are then subcloned into an appropriate expression vector for recombinant production of the SUMO-derived polypeptides.

### E. Chemical Synthesis of Polypeptides

The amino acid sequence of human SUMO1 protein has been established (e.g., GenBank Accession No. AAC50996.1 and SEQ ID NO:1). Polypeptides of known sequences, especially those of relatively short length such as the 15-55 segment of human SUMO amino acid sequence set forth in SEQ ID NO:1, may be synthesized by solid-phase peptide synthesis methods using procedures similar to those described by Merrifield et al., J. Am. Chem. Soc., 85:2149-2156 (1963); Barany and Merrifield, Solid-Phase Peptide Synthesis, in The Peptides: Analysis, Synthesis, Biology Gross and Meienhofer (eds.), Academic Press, N.Y., vol. 2, pp. 3-284 (1980); and Stewart et al., Solid Phase Peptide Synthesis 2nd ed., Pierce Chem. Co., Rockford, Ill. (1984). During synthesis, N-α-protected amino acids having protected side chains are added stepwise to a growing polypeptide chain linked by its C-terminal and to a solid support, i.e., polystyrene beads. The peptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile.

Materials suitable for use as the solid support are well known to those of skill in the art and include, but are not limited to, the following: halomethyl resins, such as chloromethyl resin or bromomethyl resin; hydroxymethyl resins; phenol resins, such as 4-(α-[2,4-dimethoxyphenyl]-Fmoc-aminomethyl)phenoxy resin; tert-alkyloxycarbonyl-hydrazidated resins, and the like. Such resins are commercially available and their methods of preparation are known by those of ordinary skill in the art.

Briefly, the C-terminal N-α-protected amino acid is first attached to the solid support. The N-α-protecting group is then removed. The deprotected α-amino group is coupled to the activated α-carboxylate group of the next N-α-protected amino acid. The process is repeated until the desired peptide is synthesized. The resulting peptides are then cleaved from the insoluble polymer support and the amino acid side chains deprotected. Longer peptides can be derived by condensation of protected peptide fragments. Details of appropriate chemistries, resins, protecting groups, protected amino acids and reagents are well known in the art and so are not discussed in detail herein (See, Atherton et al., Solid Phase Peptide Synthesis: A Practical Approach, IRL Press (1989), and Bodanszky, Peptide Chemistry, A Practical Textbook, 2nd Ed., Springer-Verlag (1993)).

### III. EXPRESSION AND PURIFICATION OF RECOMBINANT POLYPEPTIDES

Following verification of the coding sequence, a polypeptide of interest (e.g., a SUMO-derived polypeptide) can be produced using routine techniques in the field of recombinant genetics, relying on the polynucleotide sequences encoding the polypeptide disclosed herein.

### A. Expression Systems

To obtain high level expression of a nucleic acid encoding a polypeptide of interest, one typically subclones the polynucleotide coding sequence into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra. Bacterial expression systems for expressing recombinant polypeptides are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically includes a transcription unit or expression cassette that contains all the additional elements required for the expression of the desired polypeptide in host cells. A typical expression cassette thus contains a promoter operably linked to the nucleic acid sequence encoding the polypeptide and signals required for efficient polyadenylation of the transcript, ribosome binding sites, and translation termination. The nucleic acid sequence encoding the desired polypeptide is typically linked to a cleavable signal peptide sequence to promote secretion of the recombinant polypeptide by the transformed cell. Such signal peptides include, among others, the signal peptides from tissue plasminogen activator, insulin, and neuron growth factor, and juvenile hormone esterase of Heliothis virescens. If, however, a recombinant polypeptide is intended to be expressed on the host cell surface, an appropriate anchoring sequence is used in concert with the coding sequence. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as a baculovirus vector in insect cells, with a polynucleotide sequence encoding the desired polypeptide under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary. Similar to antibiotic resistance selection markers, metabolic selection markers based on known metabolic pathways may also be used as a means for selecting transformed host cells.

When periplasmic expression of a recombinant polypeptide is desired, the expression vector further comprises a sequence encoding a secretion signal, such as the E. coli OppA (Periplasmic Oligopeptide Binding Protein) secretion signal or a modified version thereof, which is directly connected to 5′ of the coding sequence of the protein to be expressed. This signal sequence directs the recombinant protein produced in cytoplasm through the cell membrane into the periplasmic space. The expression vector may further comprise a coding sequence for signal peptidase 1, which is capable of enzymatically cleaving the signal sequence when the recombinant protein is entering the periplasmic space. More detailed description for periplasmic production of a recombinant protein can be found in, e.g., Gray et al., Gene 39: 247-254 (1985), U.S. Pat. Nos. 6,160,089 and 6,436,674.

As discussed above, a person skilled in the art will recognize that various conservative substitutions can be made to a protein or its coding sequence while still retaining the biological activity of the protein. Moreover, modifications of a polynucleotide coding sequence may also be made to accommodate preferred codon usage in a particular expression host without altering the resulting amino acid sequence.

### B. Transfection Methods

Standard transfection methods are used to produce bacterial, mammalian, yeast, insect, or plant cell lines that express large quantities of a recombinant polypeptide, which are then purified using standard techniques (see, e.g., Colley et al., J. Biol. Chem. 264: 17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132: 349-351 (1977); Clark-Curtiss & Curtiss, Methods in Enzymology 101: 347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well-known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the recombinant polypeptide.

### C. Purification of Recombinantly Produced Polypeptides

Once the expression of a recombinant polypeptide in transfected host cells is confirmed, e.g., by an immunological assay, the host cells are then cultured in an appropriate scale for the purpose of purifying the recombinant polypeptide.

### 1. Purification of Recombinantly Produced Polypeptide from Bacteria

When desired polypeptides are produced recombinantly by transformed bacteria in large amounts, typically after promoter induction, although expression can be constitutive, the polypeptides may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells, e.g., by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al. and Sambrook and Russell, both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, may be inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques. For further description of purifying recombinant polypeptides from bacterial inclusion body, see, e.g., Patra et al., Protein Expression and Purification 18: 182-190 (2000).

Alternatively, it is possible to purify recombinant polypeptides from bacterial periplasm. Where the recombinant protein is exported into the periplasm of the bacteria, the periplasmic fraction of the bacteria can be isolated by cold osmotic shock in addition to other methods known to those of skill in the art (see e.g., Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

### 2. Standard Protein Separation Techniques for Purification

When a recombinant polypeptide is expressed in host cells in a soluble form, its purification can follow the standard protein purification procedure described below. This standard purification procedure is also suitable for purifying polypeptides obtained from chemical synthesis (e.g., a SUMO-derived polypeptide).

### i. Solubility Fractionation

Often as an initial step, and if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

### ii. Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of a protein of interest, e.g., a SUMO-derived polypeptide. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

### iii. Column Chromatography

The proteins of interest (such as a SUMO-derived polypeptide) can also be separated from other proteins on the basis of their size, net surface charge, hydrophobicity, or affinity for ligands. In addition, antibodies raised against a SUMO protein or a fragment thereof can be conjugated to column matrices and the corresponding polypeptide immunopurified. All of these methods are well known in the art.

It will be apparent to one of skill that chromatographic techniques can be performed at any scale and using equipment from many different manufacturers (e.g., Pharmacia Biotech).

### IV. PHARMACEUTICAL COMPOSITIONS AND ADMINISTRATION

The present invention also provides pharmaceutical compositions comprising an effective amount of a SUMO-derived polypeptide for inhibiting α-syneuclein aggregation, therefore useful in both prophylactic and therapeutic applications designed for various neurodegenerative diseases and conditions involving α-synuclein aggregation and/or amyloid fibril formation. Pharmaceutical compositions of the invention are suitable for use in a variety of drug delivery systems. Suitable formulations for use in the present invention are found in Remington's Pharmaceutical Sciences, Mack Publishing Company, Philadelphia, Pa., 17th ed. (1985). For a brief review of methods for drug delivery, see, Langer, Science 249: 1527-1533 (1990).

The pharmaceutical compositions of the present invention can be administered by various routes, e.g., oral, subcutaneous, transdermal, transnasal, intramuscular, intravenous, or intraperitoneal. The routes of administering the pharmaceutical compositions include systemic or local delivery to a subject suffering from a neurodegenerative disease at daily doses of about 0.01-5000 mg, preferably 5-500 mg, of a SUMO-derived polypeptide for a 70 kg adult human per day. The appropriate dose may be administered in a single daily dose or as divided doses presented at appropriate intervals, for example as two, three, four, or more subdoses per day.

For preparing pharmaceutical compositions containing a SUMO-derived polypeptide, inert and pharmaceutically acceptable carriers are used. The pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

In powders, the carrier is generally a finely divided solid that is in a mixture with the finely divided active component, e.g., a SUMO-derived polypeptide. In tablets, the active ingredient (the SUMO-derived polypeptide) is mixed with the carrier having the necessary binding properties in suitable proportions and compacted in the shape and size desired.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 70% by weight of the active ingredient. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active compound of a SUMO-derived polypeptide with encapsulating material as a carrier providing a capsule in which the polypeptide (with or without other carriers) is surrounded by the carrier, such that the carrier is thus in association with the compound. In a similar manner, cachets can also be included. Tablets, powders, cachets, and capsules can be used as solid dosage forms suitable for oral administration.

Liquid pharmaceutical compositions include, for example, solutions suitable for oral or parenteral administration, suspensions, and emulsions suitable for oral administration. Sterile water solutions of the active component (e.g., a SUMO-derived polypeptide) or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component (e.g., a SUMO-derived polypeptide) in the desired solvent system, and then passing the resulting solution through a membrane filter to sterilize it or, alternatively, by dissolving the sterile compound in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9, and most preferably from 7 to 8.

The pharmaceutical compositions containing the SUMO-derived polypeptide can be administered for prophylactic and/or therapeutic treatments. In therapeutic applications, compositions are administered to a patient already suffering from a neurodegenerative disease in an amount sufficient to prevent, cure, reverse, or at least partially slow or arrest the symptoms of the condition and its complications. An amount adequate to accomplish this is defined as a “therapeutically effective dose.” Amounts effective for this use will depend on the severity of the disease or condition and the weight and general state of the patient, but generally range from about 0.1 mg to about 2,000 mg of the polypeptide per day for a 70 kg patient, with dosages of from about 5 mg to about 500 mg of the polypeptide per day for a 70 kg patient being more commonly used.

In prophylactic applications, pharmaceutical compositions containing a SUMO-derived polypeptide are administered to a patient susceptible to or otherwise at risk of developing a neurodegenerative disease or disorder in an amount sufficient to delay or prevent the onset of the symptoms. Such an amount is defined to be a “prophylactically effective dose.” In this use, the precise amounts of the polypeptide again depend on the patient's state of health and weight, but generally range from about 0.1 mg to about 2,000 mg of the polypeptide for a 70 kg patient per day, more commonly from about 5 mg to about 500 mg for a 70 kg patient per day.

Single or multiple administrations of the compositions can be carried out with dose levels and pattern being selected by the treating physician. In any event, the pharmaceutical formulations should provide a quantity of a compound sufficient to effectively inhibit α-synuclein aggregation and/or amyloid fibril formation in the patient, either therapeutically or prophylactically.

### V. Therapeutic Applications Using Nucleic Acids

A variety of neurodegenerative diseases involving α-synuclein aggregation and/or amyloid fibril formation can be treated by therapeutic approaches that involve introducing into a cell a nucleic acid encoding a SUMO-derived polypeptide such that the expression of the polypeptide leads to reduced or abolished α-synuclein aggregation and amyloid fibril formation in the neuronal cells. Those amenable to treatment by this approach include a broad spectrum of conditions exemplified by Alzheimer's disease and Parkinson's disease. For discussions on the application of gene therapy towards the treatment of genetic as well as acquired diseases, see, Miller Nature 357:455-460 (1992); and Mulligan Science 260:926-932 (1993).

### A. Vectors for Nucleic Acid Delivery

For delivery to a cell or organism, a nucleic acid of the invention can be incorporated into a vector. Examples of vectors used for such purposes include expression plasmids capable of directing the expression of the SUMO-derived polypeptides in the target cell. In other instances, the vector is a viral vector system wherein the polynucleotide coding sequence is incorporated into a viral genome that is capable of transfecting the target cell. In a preferred embodiment, the coding sequence can be operably linked to expression and control sequences that can direct transcription of sequence in the desired target host cells. Thus, one can achieve reduced or abolished α-synuclein aggregation under appropriate conditions in the target cell, such as a neuronal cell.

### B. Gene Delivery Systems

As used herein, “gene delivery system” refers to any means for the delivery of a nucleic acid of the invention to a target cell. Viral vector systems useful in the introduction and expression of a SUMO-derived polypeptide include, for example, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including but not limited to Rous sarcoma virus), and MoMLV. Typically, the coding sequence for the SUMO-derived polypeptide is inserted into such vectors to allow packaging of the gene construct, typically with accompanying viral DNA, followed by infection of a sensitive host cell and expression of the polypeptide.

Similarly, viral envelopes used for packaging gene constructs that include the coding sequence for a SUMO-derived polypeptide can be modified by the addition of receptor ligands or antibodies specific for a receptor to permit receptor-mediated endocytosis into specific cells (see, e.g., WO 93/20221, WO 93/14188, and WO 94/06923).

Retroviral vectors may also be useful for introducing the SUMO-derived polypeptide of the invention into target cells or organisms. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site) (see, Mulligan, In: Experimental Manipulation of Gene Expression, Inouye (ed), 155-173 (1983); Mann et al., Cell 33:153-159 (1983); Cone and Mulligan, Proceedings of the National Academy of Sciences, U.S.A., 81:6349-6353 (1984)).

The design of retroviral vectors is well known to those of ordinary skill in the art. In brief, if the sequences necessary for encapsidation (or packaging of retroviral RNA into infectious virions) are missing from the viral genome, the result is a cis acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including, e.g., European Patent Application EPA 0 178 220; U.S. Pat. No. 4,405,712, Gilboa Biotechniques 4: 504-512 (1986); Mann et al., Cell 33:153-159 (1983); Cone and Mulligan Proc. Natl. Acad. Sci. USA 81:6349-6353 (1984); Eglitis et al. Biotechniques 6:608-614 (1988); Miller et al. Biotechniques 7:981-990 (1989); Miller (1992) supra; Mulligan (1993), supra; and WO 92/07943.

The retroviral vector particles are prepared by recombinantly inserting the desired coding sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence. As a result, the patient is capable of producing, for example, the SUMO1 protein fragment 15-55, thus reducing or abolishing unwanted α-synuclein aggregation and amyloid fibril formation.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include Crip, GPE86, PA317 and PG13 (see Miller et al., J. Virol. 65:2220-2224 (1991)). Examples of other packaging cell lines are described in Cone and Mulligan Proceedings of the National Academy of Sciences, USA, 81: 6349-6353 (1984); Danos and Mulligan Proceedings of the National Academy of Sciences, USA, 85:6460-6464 (1988); Eglitis et al. (1988), supra; and Miller (1990), supra.

### C. Pharmaceutical formulations

When used for pharmaceutical purposes, the nucleic acid encoding a SUMO-derived polypeptide is generally formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. Biochemistry 5:467 (1966).

The compositions can further include a stabilizer, an enhancer, and/or other pharmaceutically acceptable carriers or vehicles. A pharmaceutically acceptable carrier can contain a physiologically acceptable compound that acts, for example, to stabilize the nucleic acid of the invention and any associated vector. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, Pa., 17th ed. (1985).

### D. Administration of Formulations

The formulations containing a nucleic acid encoding a SUMO-derived polypeptide can be delivered to any tissue or organ using any delivery method known to the ordinarily skilled artisan. In some embodiments of the invention, the nucleic acid is formulated in mucosal, topical, and/or buccal formulations, particularly mucoadhesive gel and topical gel formulations. Exemplary permeation enhancing compositions, polymer matrices, and mucoadhesive gel preparations for transdermal delivery are disclosed in U.S. Pat. No. 5,346,701.

The formulations containing the encoding nucleic acid are typically administered to a cell. The cell, such as a neuronal cell, can be provided as part of a tissue or as an isolated cell, such as in tissue culture. The cell can be provided in vivo, ex vivo, or in vitro.

The formulations can be introduced into the tissue of interest in vivo or ex vivo by a variety of methods. In some embodiments of the invention, the encoding nucleic acid is introduced into cells by such methods as microinjection, calcium phosphate precipitation, liposome fusion, ultrasound, electroporation, or biolistics. In further embodiments, the nucleic acid is taken up directly by the tissue of interest.

In some embodiments of the invention, the encoding nucleic acid is administered ex vivo to cells or tissues explanted from a patient, then returned to the patient. Examples of ex vivo administration of therapeutic gene constructs include Nolta et al., Proc Natl. Acad. Sci. USA 93(6):2414-9 (1996); Koc et al., Seminars in Oncology 23(1):46-65 (1996); Raper et al., Annals of Surgery 223(2):116-26 (1996); Dalesandro et al., J. Thorac. Cardi. Surg., 11(2):416-22 (1996); and Makarov et al., Proc. Natl. Acad. Sci. USA 93(1):402-6 (1996).

Effective dosage of the formulations will vary depending on many different factors, including means of administration, target site, physiological state of the patient, and other medicines administered. Thus, treatment dosages will need to be titrated to optimize safety and efficacy. In determining the effective amount of the vector to be administered, the physician should evaluate the particular nucleic acid used, the disease state being diagnosed; the age, weight, and overall condition of the patient, circulating plasma levels, vector toxicities, progression of the disease, and the production of anti-vector antibodies. The size of the dose also will be determined by the existence, nature, and extent of any adverse side-effects that accompany the administration of a particular vector. To practice the present invention, doses ranging from about 10 ng-1 g, 100 ng-100 mg, 1 μg-10 mg, or 30-300 μg encoding nucleic acid per patient are typical. Doses generally range between about 0.01 and about 50 mg per kilogram of body weight, preferably between about 0.1 and about 5 mg/kg of body weight or about 10⁸-10¹⁰ or 10¹² viral particles per injection. In general, the dose equivalent of a naked nucleic acid from a vector is from about 1 μg-100 μg for a typical 70 kg patient, and doses of vectors which include a retroviral particle are calculated to yield an equivalent amount of a nucleic acid encoding a SUMO-derived polypeptide.

### VI. KITS

The invention also provides kits for suppressing α-synuclein aggregation or treating a neurodegenerative condition involving α-synuclein aggregation and/or amyloid fibril formation by administering a SUMO-derived polypeptide or nucleic acid encoding the polypeptide according to the method of the present invention. The kits typically include a first container that contains a pharmaceutical composition having an effective amount of a SUMO-derived polypeptide, optionally with a second container containing a neuroprotective agent, for example, an antioxidant such as acetylcysteine, crocin, delta 9-tetrahydrocannabinol (THC), fish oil, minocycline, pyrroloquinoline quinone (PQQ), resveratol, vinpcetine, and vitamin E, or an NMDA receptor stimulator, for example, caffeine, nicotine, and selegiline. In some cases, the kits will also include informational material containing instructions on how to dispense the pharmaceutical composition, including description of the type of patients who may be treated (e.g., a person suffering from a neurodegenerative disease such as Alzheimer's disease or Parkinson's disease), the schedule (e.g., dose and frequency of administration) and route of administration, and the like.

### EXAMPLES

The following examples are provided by way of illustration only and not by way of limitation. Those of skill in the art will readily recognize a variety of non-critical parameters that could be changed or modified to yield essentially the same or similar results.

### Example 1

### INTRODUCTION

Human α-synuclein is a 14-kDa protein encoded by the SNCA gene (4q21-q23) (Chen et al., 1995). Abnormal α-synuclein aggregates, or Lewy bodies, are pathological hallmarks of a number of neurodegenerative diseases, most notably Parkinson's disease (PD). The primary sequence of α-synuclein (140 aa) can be subdivided into three regions with distinct properties: The N-terminal region (residues 1-60), the non-amyloid β component (NAC) domain (residues 61-95) and the C-terminal region (residues 96-140). N-terminal missense mutations (A30P, E46K, H50Q, G51D, A53E and A53T) (Appel-Cresswell et al., 2013; Kruger et al., 1998; Lesage et al., 2013; Pasanen et al., 2014; Proukakis et al., 2013; Zarranz et al., 2004) in α-synuclein protein were reported as genetic cause of familial PD. A central segment of α-synuclein termed NACore (residues 68-78) is responsible for both the amyloid formation and cytotoxicity of α-synuclein (Rodriguez et al., 2015). And a hydrophobic stretch, residues 74-82, is essential for α-synuclein filament assembly (Giasson et al., 2001; Guerrero-Ferreira et al., 2018). C-terminal region, serving as solubilizing domain, is responsible for thermostability (Park et al., 2002) and chaperone-like function (Souza et al., 2000). This region also regulates amyloid aggregation because C-terminally truncated α-synuclein aggregates faster than full length version (Crowther et al., 1998). The quenched hydrogen/deuterium exchange NMR data indicated α-synuclein fibril core consisting of five β-strands (residues 37-43, 52-59, 62-66, 68-77, and 90-95) and solid-state NMR confirmed the presence of β-sheet secondary structure (Vilar et al., 2008).

Unlike normal functional α-synuclein, aggregated α-synuclein transfer between neuron to neuron in a prion-like manner in vitro and in vivo (Angot et al., 2012; Aulic et al., 2014; Desplats et al., 2009; Hansen et al., 2011). After uptake into neuronal cells, α-synuclein fibrils recruit endogenous soluble α-synuclein, converting it into Lewy body-like inclusions (Luk et al., 2009). Multiple cellular functions are disrupted by toxicity induced by α-synuclein aggregation, for example synaptic-vesicle trafficking, mitochondria function, regulation of organelle dynamics and autophagy or lysosomal pathway (Wong and Krainc, 2017). Notably the early prominent death of dopaminergic neurons in the substantia nigra pars compacta (SNpc) results in dopamine loss in the basal ganglia leading to the onset of clinical Parkinson's motor symptoms (Kalia and Lang, 2015). Although which form, oligomer or fibril, is more toxic is still a controversial topic, reducing their formation is a comprehensive therapeutic strategy to slow down α-synuclein related disease progression or delay disease onset.

Recent findings have suggested that sumoylation plays an important role in neurodegenerative diseases including PD (Krumova and Weishaupt, 2013). Sumoylation is a post-translational modification which involves the addition of a small ubiquitin-like modifier (SUMO) to a specific lysine on proteins with the help of three enzymes-the E1 activating enzyme, E2 conjugating enzyme and E3 ligase. There are 4 kinds of SUMO proteins in mammals: SUMO1 to SUMO4. Sumoylation has been shown to affect protein stability, direct cellular localization, and alter enzymatic activity by creating new interaction surfaces or blocking existing interaction domains (Geiss-Friedlander and Melchior, 2007). There is growing evidence that sumoylation is essential in the development of the central nervous system (CNS) and in neuron-specific functions. For instance, synaptic transmission, plasticity, hippocampal neuron excitability and neuron maturation (Loriol et al., 2012; Martin et al., 2007; Plant et al., 2011). In addition, it has been found that sumoylation relieves some neurodegenerative diseases by increasing aggregated protein solubility (Janer et al., 2010; Krumova et al., 2011; Mukherjee et al., 2009). In addition, the ability of SUMO paralogues non-covalently bind to other proteins containing SUMO-interaction motif (SIM) may affect sumoylation and the following cell functions. SIM normally consisting of a Ser-Xaa-Ser motif flanked by hydrophobic and acidic amino acids forms a β-strand that could bind to the β₂-strand of SUMO in parallel or antiparallel orientation. Apart from β₂-strand, several residues locate in the α-helix also involved in the interaction (Hecker et al., 2006; Minty et al., 2000).

In this study, it was unexpectedly observed that SUMO1 variants could directly delay or abrogate α-synuclein aggregation without the formation of the isopeptide bond associated with sumoylation. Since therapeutic peptides have several advantages over proteins (Mason and Fairlie, 2015), it was explored whether a peptide core would be able to suppress aggregation similarly. If SUMO1 is truncated into effective peptide, it will not affect the physiological function of SUMO1 in human body. As a peptide derived from existing protein in human body, it would be less immunogenic. Then the functional fragments, β₂-strand and α-helix, involved in SUMO-SIM interactions, were preserved, and SUMO1 was truncated into a small peptide-SUMO1(15-55) or SUMO1(31-55).

Microscale thermophoresis (MST) data indicated that SUMO1(15-55) interacted with the β1, β4 strands of α-synuclein (Table 2). The suppression effect of SUMO1(15-55) was sequentially tested in vitro, cell-based model and α-synuclein transgenic Drosophila model, and the results showed that SUMO1(15-55) significantly inhibited α-synuclein aggregation, reduced its toxicity and ameliorated disease symptoms. This study provides information that can ultimately lead to a new treatment for α-synuclein aggregation induced PD and/or synucleinopathies.

### MATERIALS AND METHODS

### Protein Expression and Purification

Expression and purification of recombinant α-synuclein was conducted as previously described (Giasson et al., 1999). In brief, the gene encoding human wild-type α-synuclein was amplified by PCR with corresponding primers (Table 3) and then inserted into the NcoI and XhoI sites of pET-28a vector (Novagen). The construct was expressed with Escherichia coli BL21(DE3) in LB medium. Protein expression was induced with 0.5 mM isopropyl β-D-1-thiogalactoside (IPTG) at OD 0.4-0.6 for 4 h at 37° C. Following centrifugation at 14,000 rpm, the cell pellet was resuspended in 20 mM HEPES, pH 7.4 and lysed by sonication. Insoluble fraction was removed by centrifugation and soluble fraction was boiled for 10 min in water bath. The heat-stable supernatant was filtered through a 0.22 μM filter (Satorius) and loaded on High Q anion exchange column (5 ml, Bio-Rad). After gradient elution, all fractions were examined by 15% SDS-PAGE and purified fractions were dialyzed against 20 mM HEPES, 150 mM NaCl, pH 7.4 at 4° C. overnight. Then protein was concentrated using Vivaspin columns (MWCO 3 kDa, GE Healthcare), centrifuged at 14,000 rpm for 15 min to remove potential aggregates. The concentrated α-synuclein was further purified with SEC 70 high-resolution size exclusion column (24 ml, Bio-Rad) with 20 mM HEPES, 150 mM NaCl, pH 7.4 as running buffer. The protein concentration was determined photometrically at 280 nm using an extinction coefficient of ε=5960 M⁻¹ cm⁻¹.

Different coding regions of human small ubiquitin-like modifier 1 (SUMO1) were amplified by PCR with corresponding primers (Table 3). Amplified fragments were subcloned into the NdeI and XhoI sites of pET-28a vector (Novagen), and constructs were used to transform Escherichia coli BL21(DE3). Expression cultures were maintained in LB medium. Protein expression was induced with 0.5 mM isopropyl β-d-thiogalactoside (IPTG) at OD 0.4-0.6 for 4 h at 37° C. Following centrifugation at 14,000 rpm, the cell pellet was resuspended in 20 mM HEPES, 500 mM NaCl, pH 7.4 and then lysed by sonication. Supernatant was filtered through a 0.22 μM filter (Satorius) and loaded on nickel-sepharose column (GE Healthcare). N-terminal His-tag was removed by thrombin (GE Healthcare) according to instructions. After gradient elution, all fractions were examined by 15% SDS-PAGE and purified fractions were concentrated using Vivaspin columns (MWCO 3 kDa, GE Healthcare), followed by gel filtration over a SEC 70 high-resolution size exclusion column (24 ml, Bio-Rad) with 20 mM HEPES, 150 mM NaCl, pH 7.4 as running buffer. The protein concentration was determined photometrically at 280 nm using an extinction coefficient of ε=4470 M⁻¹ cm⁻¹.

### Cross-Linking Reaction

Proteins were dialyzed against phosphate buffered saline (PBS), pH 7.4. Subsequently, cross-linking experiment was conducted following the instructions. Sulfo-GMBS (N-[γ-maleimidobutyloxy] succinimide ester) (7.3 Å spacer arm, Thermo Fisher Scientific) was added into α-synuclein and the mixture was incubated for 30 min at ambient temperature. Followed by removing of excess cross-linker using a desalting column (7K MWCO, Thermo Fisher Scientific), dialyzed SUMO1(15-92) was added to desalted α-synuclein and the mixture was further incubated for 30 min. Samples were separated with 15% SDS-PAGE. Upshifted bands were harvested and trypsinized into random digested peptides and processed by Nano liquid chromatography-Fourier transform ion cyclotron resonance mass spectrometry (NanoLC-FTICR-MS) (Bruker Daltonics Apex Ultra 7.0 T with Dionex Ultimate 3000 Nano LC). External calibration of the instrument was performed to a mass accuracy of less than 1 ppm with direct infusion of Bovine Serum Albumin (BSA) tryptic peptides prior to the experiment. Cross-linked peptides were assigned by matching the experimental ion masses with the masses of all theoretically digested fragments plus the molecular weight change caused by cross-linker. Data was processed using SequenceEditor tool in BioTools software (Bruker Daltronics).

### Fibrillation Assay

The reaction contained the mixture of 70 μM α-synuclein and different concentrations of SUMO1 variants, 40 μM Thioflavin T (ThT) (Sigma-Aldrich), 0.4 mM SDS in reaction buffer (20 mM HEPES, 150 mM NaCl, pH 7.4). Reactions were performed in quadruplicate in a black clear-bottomed 96-well plate (Nunc, Thermo Fisher Scientific) with a final volume of 150 μL. The plate was sealed with sealing membrane (Thermo Fisher Scientific) and loaded into an Infinite M1000 plate reader (Tecan), incubated at 37° C. without agitation. The fluorescence was detected at 30 min intervals, with excitation at 450 nm and with emission at 485 nm. Protein samples were analyzed after fibrillation assay using size-exclusion chromatography on a Superdex 200 GL 5/150 column (3 ml, GE Healthcare) in reaction buffer.

### Transmission Electron Microscopy

Samples were spotted directly to formvar support films mounted on 200-mesh copper grids (Electron Microscopy Sciences), incubated for 5 min, and then rinsed twice with distilled water. Negative staining was performed by staining with 2% uranyl acetate for 2 min. After air drying, specimens were inspected with a Hitachi H-7650 transmission electron microscope operated at 80 keV.

### Circular Dichroism Spectroscopy

Protein samples were incubated at 37° C. for designed time points and then diluted in a buffer of 20 mM Tris, 150 mM NaCl, pH 7.4 for CD measurement. The final concentration of α-synuclein was fixed at 20 μM. The spectra of the solution samples were measured from 250 to 200 nm using a 1 mm pathlength quartz cuvette on a JASCO J-810 CD spectrometer at 25° C. The band width was set to 1 nm, data pitch was 0.1 nm and scan speed was 50 nm/min. Raw data were processed by smoothing and subtraction of buffer spectra.

### Microscale Thermophoresis

Protein targets were labelled using the Alexa Fluor 647 NHS ester dye (Thermo Fisher Scientific). The labelling reaction was performed according to the manufacturer's instructions. Unreacted dye was removed with the dye removal column supplied in the Protein Labelling Kit RED-NHS (NanoTemper Technologies) or desalting column (Bio-Rad). The purity was monitored by measuring the ratio of protein to dye (for example, spectroscopically by measuring absorption at 280 nm for protein and 650 nm for the dye; molar absorbance: 239,000 M⁻¹ cm⁻¹) after the clean-up procedure. The labelled targets were adjusted to appropriate concentration for detection. The ligands were dissolved in reaction buffer and a series of 16 1:1 dilutions was prepared. For the measurement, each ligand dilution was mixed with one volume of labelled protein target. After 10 min incubation, the samples were loaded into standard or premium Monolith NT.115 Capillaries (NanoTemper Technologies). MST was measured using a Monolith NT.115 instrument (NanoTemper Technologies) at an ambient temperature of 25° C. Instrument parameters were adjusted to 20% LED power and medium MST power. Data of three independently pipetted measurements were analyzed (MO.Affinity Analysis software version 2.1.3, NanoTemper Technologies) using the signal from an MST-on time of 5 s.

### Preparation for Aged α-Synuclein Samples in the Absence and Presence of SUMO1(15-55)

Purified α-synuclein in 20 mM HEPES, 150 mM NaCl, pH 7.4 was incubated at a concentration of 70 μM at 37° C. for 5 days in the absence and presence of stoichiometric amount of SUMO1(15-55). For NACore samples, lyophilized NACore peptide was dissolved in distilled water at a concentration of 500 μM in the absence and presence of stoichiometric amount of SUMO1(15-55). Samples were shaken at 37° C. for 3 days with continuous shaking at 500 rpm in a Thermomixer (Eppendorf).

### MTT Cell Viability Assay

SH-SY5Y neuroblastoma cells (ATCC CRL-2266) were cultured in 96-well tissue culture plate at a density of 2,000 cells per well to a final volume of 100 μL of media (DMEM/F-12, 10% fetal bovine serum, 1% penicillin-streptomycin) and incubated for 24 h. Following addition of corresponding protein samples, cells were further incubated for 24 h. Then, cells were treated with 3-(4,5-dimethylthiazol-2-yl)-2,5-diphenyltetrazolium bromide (MTT) solution (0.5 mg/mL). After 4 h incubation, media was carefully removed. Cells were lyzed with 100 μL DMSO followed by measuring the absorbance at 540 nm in an Infinite M1000 plate reader (Tecan). All cultures were maintained at 37° C. in a 5% CO2 humidified atmosphere and never exceeded 25 passages. The data was normalized to the value of cells treated with reaction buffer. Experiments were repeated at least three times with similar results.

### Exogenous α-Synuclein Fibril Internalization in SH-SY5Y

SH-SY5Y neuroblastoma cells (ATCC CRL-2266) were cultured in 12-well plate (SPL Life Sciences) at the density of 100,000 cells per well. Then, cells were treated with Alexa 488 fluorescent-tagged aggregation samples for 3 h and the final concentration of α-synuclein was fixed at 0.5 μM for each group. Aggregations samples were sonicated prior to internalization for 1.5 h in water bath. To remove extracellular aggregation samples, cells were washed three times with PBS, trypsinized, then reseeded and cultured for 20 h followed by confocal microscopy and flowcytometry.

### Biomolecular Fluorescence Complementation (BiFC)

SH-SY5Y neuroblastoma cells (ATCC CRL-2266) were cultured in 35 mm glass bottom petri dish (MatTek) at the density of 100,000 cells per dish. Cells were transfected with corresponding plasmids using Lipofectamine 2000 (Thermo Fisher Scientific) following standard protocol. Live cells were imaged 24 h after transfection.

### Confocal Microscopy

For nuclear and cell membrane staining, Hoechst 33342 (Thermo Fisher Scientific) and Alexa 647 conjugated wheat germ agglutinin (WGA) (Thermo Fisher Scientific) were added directly to the culture medium and incubated in dark for 10 min before imaging. Live-cell imaging were acquired on Leica TCS SP8 confocal microscope with 63X oil immersion objective.

### Flowcytometry

For sample preparation, SH-SY5Y cells were washed with PBS, trypsinized, pelleted, resuspended in ice-cold PBS and strained through a nylon net filter (Millipore). Per sample, 100,000 cells were analyzed on a BD FACSVerse (BD Biosciences) with excitation at 488 nm. Raw data analysis was performed using the FlowJo software (FlowJo LLC).

### Larval Feeding

Third-instar larvae were fed with 2% sucrose solution supplemented with 100 μM, 200 μM or 400 μM of SUMO1(15-55) for 2 h respectively and then continued to culture in non-drug containing food at 25° C. (Chau et al., 2006).

### Pseudopupil Assay

Pseudopupil assay was performed 3-4 days after eclosion as previously described (Berger et al., 2005). Images were acquired by a SPOT Insight CCD camera with a 60X oil objective controlled by the SPOT Advanced software (Diagnostic Instruments Inc.). For each condition, at least 650 ommatidia from 35-40 flies obtained from three independent crosses were used to calculate the average number of rhabdomeres per ommatidium.

### Climbing Assay

Climbing assay was performed according to previously described (Feany and Bender, 2000). Briefly, a group of 10 flies were placed in individual tubes for a total of four tubes. Flies were gently tapped to the bottom of the tube, and the number of flies that could climb a height of 2 cm within 15 s was recorded. For each condition, a minimum of 60 flies obtained from three independent crosses were tested. Each trial was repeated five times. All flies were maintained at 25° C. during whole process of the behavioural assay.

### Western Blot Analysis

At the end of climbing assay, Drosophila heads for each group were homogenized and boiled for 10 min. Debris were removed by centrifugation at 15,000 rpm for 10 min at 4° C. Supernatants were subjected to 15% SDS-PAGE for analysis. Proteins were transferred to nitrocellulose membranes (Bio-Rad), blocked in 5% Blocking-Grade Blocker (Bio-Rad) in PBS with 0.1% Tween 20 (Sigma-Aldrich), and immunoblotted according to standard procedures. The following primary antibodies were used: anti-tyrosine hydroxylase (1:1000; Millipore), anti-α-synuclein (clone 42, 1:1000; BD Biosciences), anti-GAPDH (Clone 6C5, 1:5000; Thermo Fisher Scientific). The appropriate horseradish peroxidase-conjugated secondary antibodies were applied, and signals were measured by chemiluminescence (Bio-Rad). All immunoblots were repeated at least three times with similar results.

### Results

### Design of the Short Therapeutic Peptide for α-Synuclein Aggregation

The initial studies focused on examining the ability of SUMO1(1-97) to inhibit α-synuclein aggregation. In order to expose the effective regions, SUMO1(15-92) was produced by truncating both N- and C-terminal floppy tails. At the same time, the inventors noted a negatively charged loop consisting of three continuous glutamic acids (residues 78-80, EEE) and decided to explore the impact of mutating these three residues to lysines (SUMO1(15-92)-KKK) to investigate significance of electrostatics on the aggregation suppression. Notably, this EEE sequence forms salt bridges with K23 on β₁-strand (residues 21-26, YIKLKV). It was thus reasoned, if this salt bridge interaction was important for stability then mutating it should alter its ability to suppress α-synuclein aggregation. The SUMO1(15-92)-KKK mutant would also allow us to evaluate the role of overall protein charge on α-synuclein aggregation suppression. These changes shift the isoelectric point from 6.47 into 9.41, making SUMO1(15-92)-KKK from electronegative into electropositive at neutral pH environment.

The fibrillation assays indicate that SUMO1(15-92) and SUMO1(15-92)-KKK both directly inhibited α-synuclein aggregation while SUMO1(1-97) could barely inhibit the aggregation. These data confirm that as expected, the N-terminal and C-terminal tails of SUMO1 are not important for α-synuclein aggregation suppression, and indeed hinder this ability. Moreover, the similar results for the SUMO1(15-92)-KKK mutation suggest that the overall charge is not a factor in the observed suppression, and nor the salt-bridge between residues 21-26 (YIKLKV) and residues 78-80 (EEE).

To further reduce the size of the SUMO1 construct to solely the binding region, the last two β-strands (β₄, β₅) were both truncated. Only the first two β-strands (β₁, β₂) and the α-helix containing residues involved in SUMO-SIM interactions were preserved yielding a shorter construct SUMO1(15-55).

### Suppression Effect of SUMO1(15-55) on the Aggregation of α-Synuclein

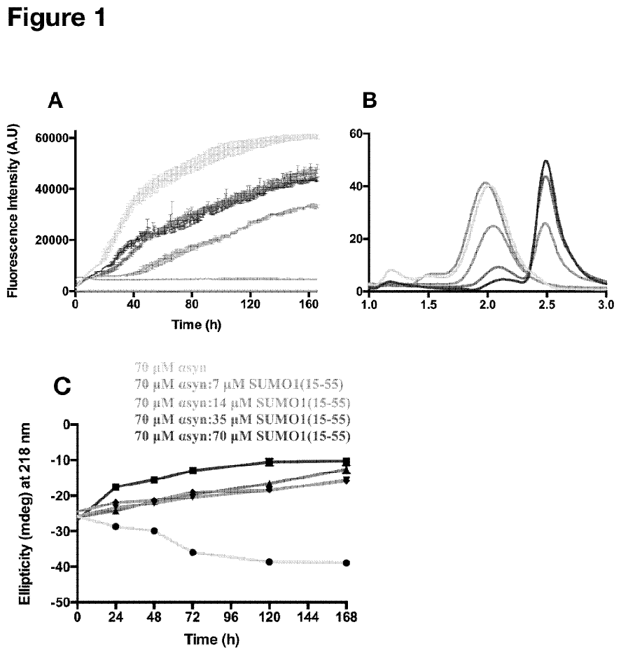

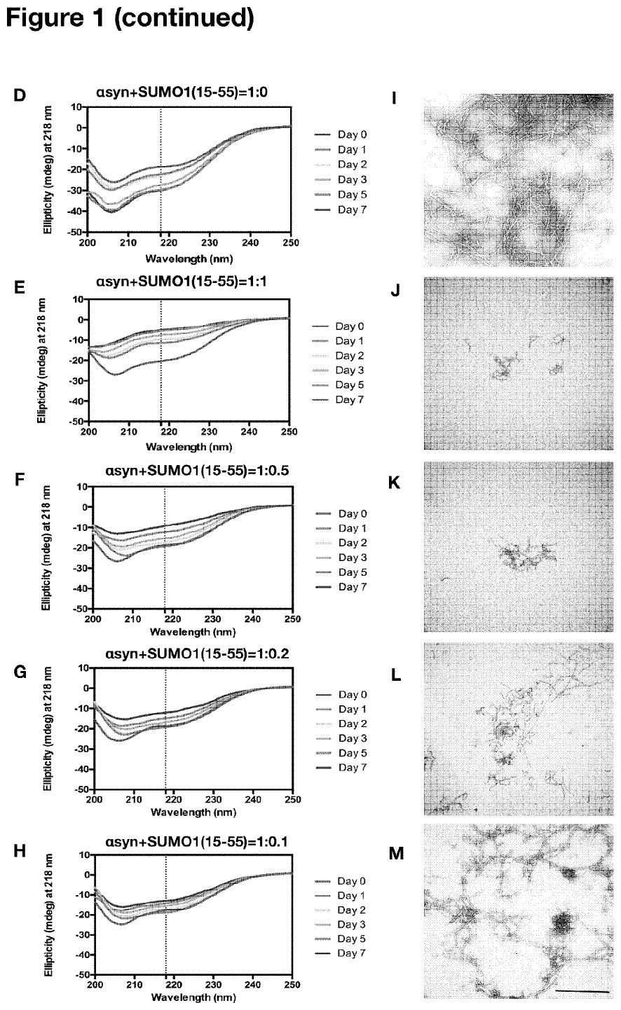

The potency of SUMO1(15-55) to inhibit α-synuclein aggregation was initially detected by Thioflavin T (ThT), which is widely used to quantify the misfolded protein aggregates. This dye displays enhanced fluorescent signals, when it binds to β-sheet rich structures in amyloid aggregates (Groenning, 2010). Sodium dodecyl sulfate (SDS) was added to mimic membrane environments and serve as nucleation scaffold to stimulate α-synuclein aggregation (Giehm et al., 2010). In fibrillation assay, SUMO1(15-55) completely inhibited α-synuclein aggregation at equimolar ratios and delayed aggregation at sub-stoichiometric levels within 7 days experiment (FIG. 1A). Consistent with fibrillation assay, TEM revealed a distinct reduction in both length and abundance of the amyloid fibrils at all fibrillation assay samples incubated with SUMO1(15-55) (FIG. 1I-M).

To explore the sub-stoichiometric inhibition mechanisms, protein samples from fibrillation assay were analyzed by size-exclusion chromatography. The results indicated that equimolar mount of SUMO1(15-55) completely maintained α-synuclein in monomeric state and sub-stoichiometric ratios of SUMO1(15-55) only kept part of α-synuclein staying in monomeric state (FIG. 1B). Also, circular dichroism (CD) was adopted to monitor secondary structure changes involved in fibrillation. As shown in FIG. 1C, the accumulation of β-sheet (an increase in the negative ellipticity at 218 nm) and the reduction of random coil (a decrease in the negative ellipticity at 200 nm) in α-synuclein CD spectrum were clearly inhibited by SUMO1(15-55) (FIG. 1D-H). Nevertheless, SUMO1(15-55) failed to reverse aggregation process (data not shown).

### Attenuation Effect of SUMO1(15-55) on Aggregated α-Synuclein Induced Cytotoxicity

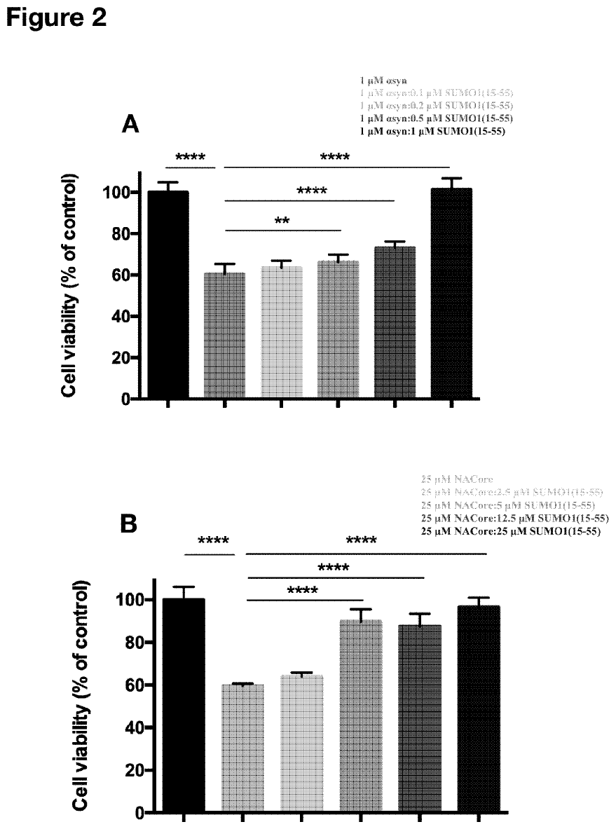

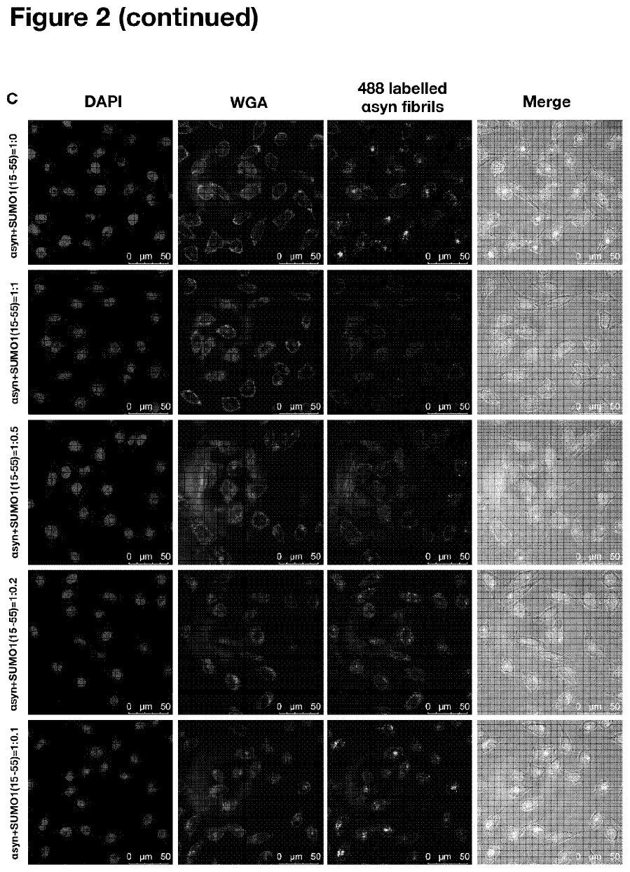

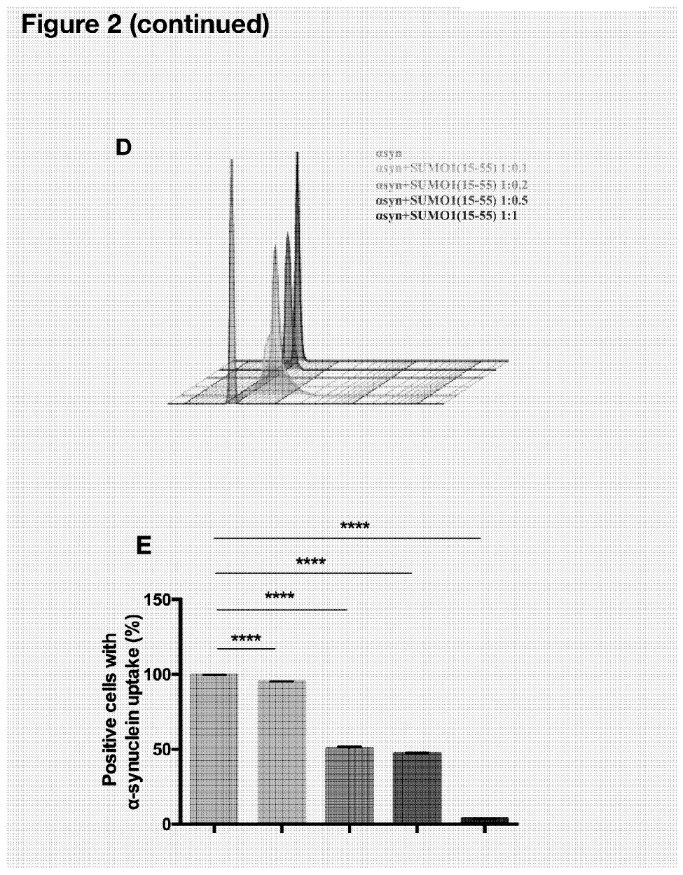

MTT assays were performed to determine whether SUMO1(15-55) could decrease aggregated α-synuclein or NACore induced cytotoxicity by measuring the viability of SH-SY5Y cells treated with α-synuclein or NACore samples aged in the absence and presence of SUMO1(15-55). Aggregated α-synuclein and NACore both exerted significant cytotoxicity to SH-SY5Y cells, however, the cellular viability was improved when aged samples were incubated in the presence of SUMO1(15-55). The rescue ability was dependent on the concentration of SUMO1(15-55), with complete rescue at equimolar ratio and partial rescue at substoichiometric amounts (FIG. 2A-B). Corresponding concentration of SUMO1(15-55) alone did not affect cell viability (data not shown).

It was further explored whether the protective effect was related to exogenous α-synuclein fibril internalization. Using confocal microscopy, the uptake of fluorescently labelled fibrils could be observed after 24h post treatment (FIG. 2C). The proportion of cells containing labelled α-synuclein fibrils was 99.6%, with significantly lower proportion of positive cells for SUMO1(15-55) treated groups in a dose dependent manner (3.4%, 47.1%, 50.7%, 95.1%) (FIG. 2D-E).

To Validate the Interaction Between SUMO1(15-55) and α-Synuclein within Neuronal Cells

Direct protein-protein interaction between α-synuclein and SUMO1(15-55) was detected after confirmation of suppression effect in vitro. Morell et al. (2008) reported an approach to visualize and quantitate in vivo weak protein interactions by coupling biomolecular fluorescence complementation (BiFC) and flowcytometry. Specifically, α-synuclein and SUMO1(15-55) were separately fused to split Venus, a modified yellow fluorescent reporter (VN173 and VC155) (Shyu et al., 2008). The interaction brings the two halves of the split Venus into close proximity allowing for reconstitution of the intact yellow fluorescent protein. Hence, the appearance of a yellow fluorescent signal is correlated with protein-protein interaction between α-synuclein and SUMO1(15-55).

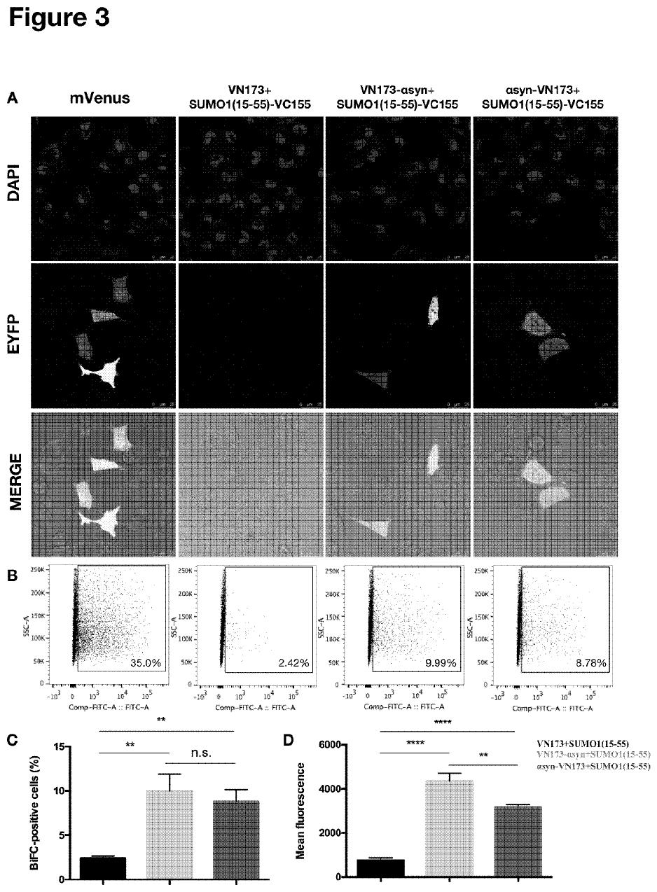

α-Synuclein and SUMO1(15-55) were coexpressed with the complementary fluorescent fragments in SH-SY5Y cells. Corresponding control groups were set at the same time to ensure the fluorescent signal is caused specifically by the binding between target proteins. The yellow fluorescent signal was initially visualized under confocal microscopy. As shown in FIG. 3A, fluorescent signal was specific to α-synuclein-SUMO1(15-55) complex formation and background fluorescence was low. Flowcytometry verified the confocal results that the percentage of BiFC-postive cells was significantly higher than negative control group (FIG. 3C), and the mean fluorescence value of experiment group, directly related to the interaction strength (Morell et al., 2008), was significantly higher than negative control groups (FIG. 3D). This fact suggested the conjugated α-synuclein and SUMO1(15-55) helped the two split halves of yellow fluorescent protein, which cannot interact with each other or the conjugated protein, reconstitute into intact form and emitted fluorescence owing to the protein-protein interaction. Also, the conjugate direction of split Venus (VN173) to α-synuclein makes no difference to the interaction according to the mean fluorescence value. Identification of around 9% cells in experimental groups containing two halves of the split Venus as BiFC positive is consistent with the fact that around 35% cells were transfected with intact Venus plasmid in positive control group (FIG. 3B).

### Neuroprotective Effect of SUMO1 (15-55) in WT α-Synuclein Transgenic Drosophila Model.

To assess the protective effect of SUMO1(15-55) in vivo, a typical animal Parkinson's disease model (Auluck and Bonini, 2002; Auluck et al., 2005; Outeiro et al., 2007) was adopted. The expression of α-synuclein was under the control of upstream activating sequence (UAS) for the yeast transcription factor GAL4 (GAL4-UAS, bipartite expression system) and directed by tissue- or cell-type-specific drivers.

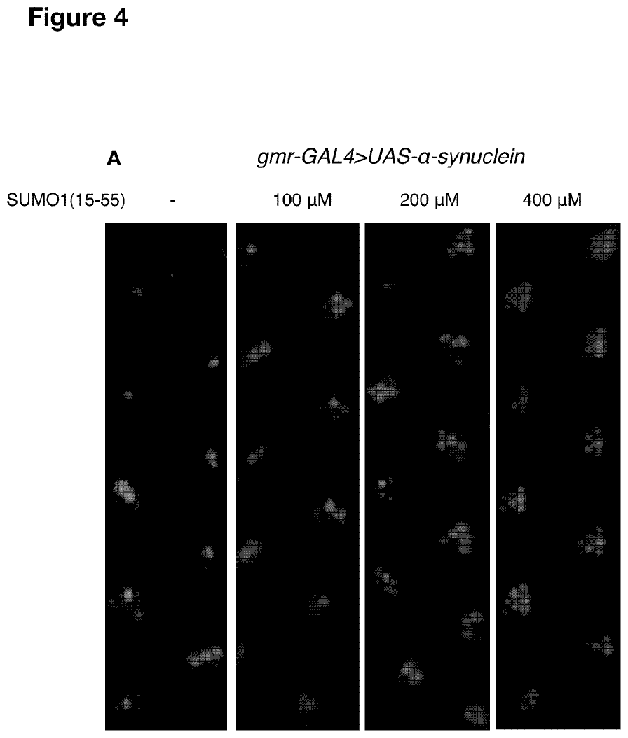

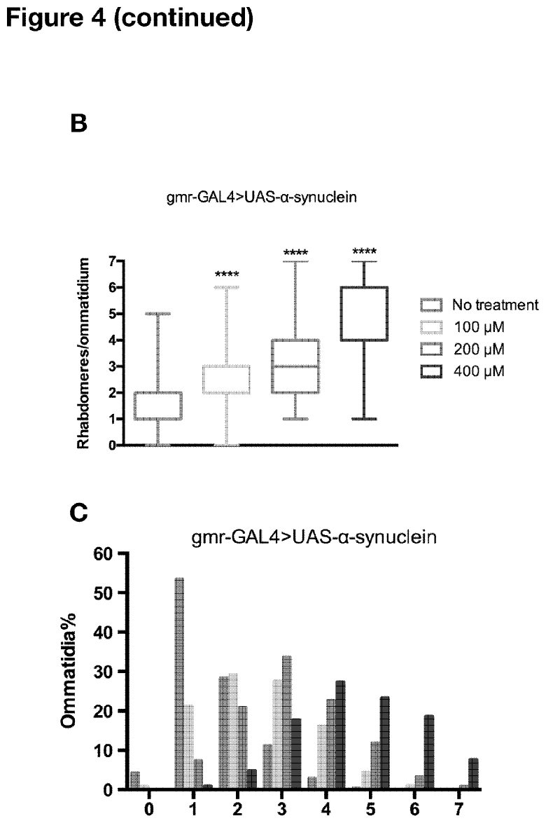

Pseudopupil assay quantitates the neurodegeneration by counting the rhabdomeres in each ommatidium (Marsh et al., 2003). Consistent with previous studies (Feany and Bender, 2000), specific expression of α-synuclein in the eye of Drosophila using gmr-GAL4 driver caused retinal degeneration. In contrast to no treatment control group, SUMO1(15-55) treatment groups significantly ameliorated the neurodegeneration process induced by α-synuclein in a dose-dependent manner (FIG. 4).

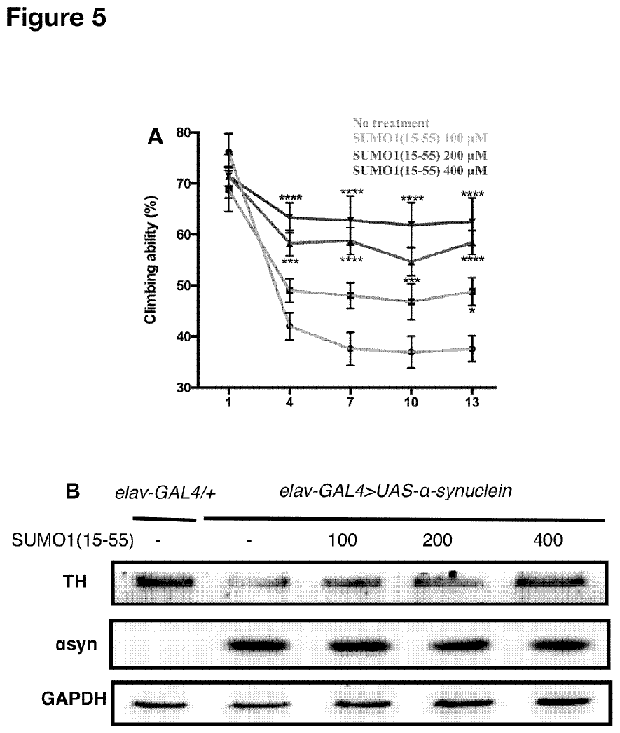

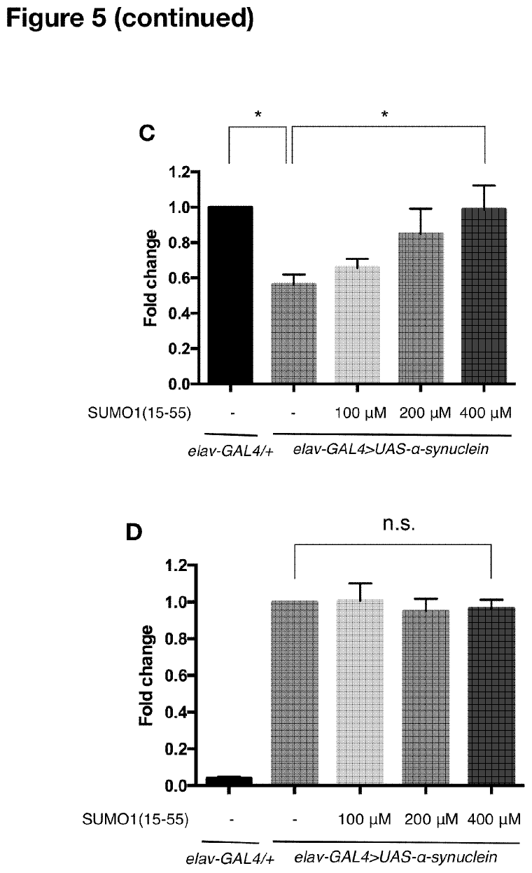

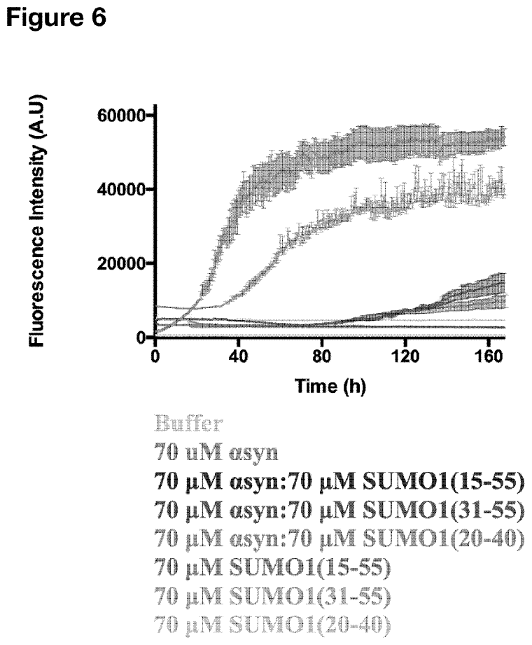

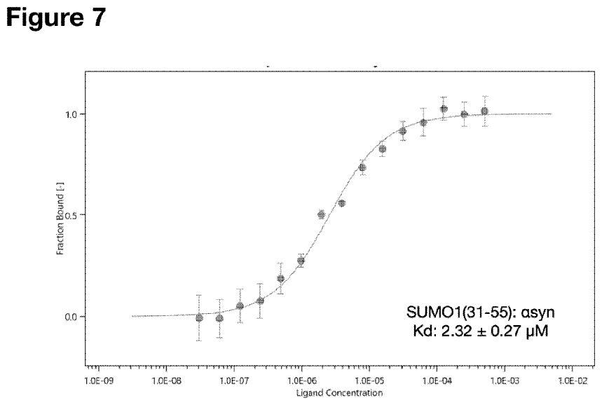

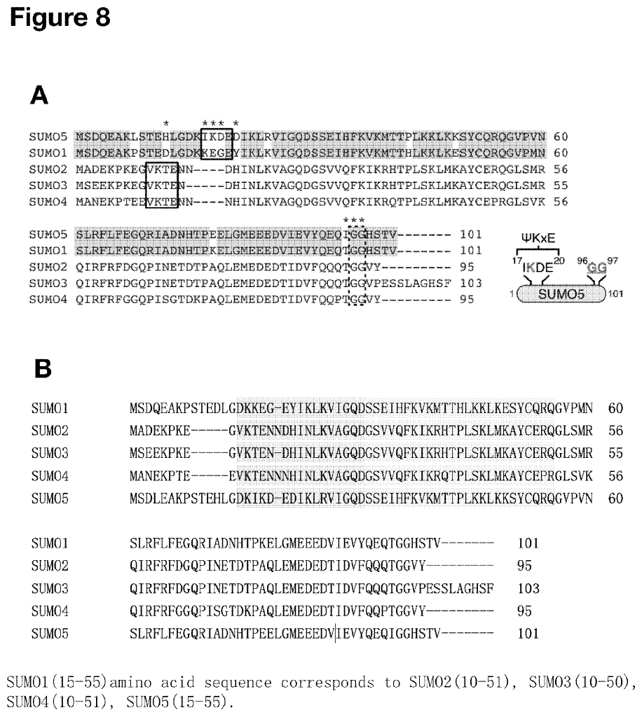

Pan-neural expression of α-synuclein using elav-GAL4 driver induced age-dependent negative geotactic climbing ability impairment in Drosophila (Feany and Bender, 2000). Consistent with previous reports, α-synuclein transgenic Drosophila showed significantly decline in negative geotactic climbing response starting from 4 days after eclosion. However, rearing the transgenic Drosophila in SUMO1(15-55) solution during disease onset stage (larval stage) significantly suppressed the progressive loss of motor function in a dose-dependent manner during the following climbing assay (FIG. 5A).

Western blot analysis was performed to further investigate the potential mechanisms of this neuroprotective effect. The expression level of α-synuclein protein was not affected by SUMO1(15-55), however, the amount of tyrosine hydroxylase (TH) is correlated with the dosage of SUMO1(15-55) (FIG. 5B-D). Taken together, SUMO1(15-55) provided neuroprotection against α-synuclein toxicity by rescuing dopaminergic neurons instead of reducing α-synuclein production.

### Exploring the Binding Regions Involved in the Interaction

After confirmation of the suppression effect in vitro and in vivo, cross-linking reaction and Nano liquid chromatography-Fourier transform ion cyclotron resonance mass spectrometry (NanoLC-FTICR-MS) were conducted to identify the interaction regions involved in the suppression. With SUMO1(15-92) construct, several strong upshifted bands were detected and then analyzed by NanoLC-FTICR-MS. Human α-synuclein contains fifteen lysines (K6, K10, K12, K21, K23, K32, K34, K43, K45, K58, K60, K80, K96, K97, K102), and SUMO1(15-92) only has one cysteine (C52) can be cross-linked. Also C52 is close to the residues involved in SUMO-SIM interactions (Hecker et al., 2006), which makes it an ideal target to investigate the binding regions on α-synuclein. In theory 15 kinds of cross-linked dipeptides can be formed. In fact, only part of lysine residues (K34, K43, K45, K58, K96) in α-synuclein, mainly locating on the β1 and β2 strand, were detected cross-linking with C52 in SUMO1(15-92) (Table 1). All these facts indicate that the inhibition mechanisms may relate to the binding of SUMO1(15-92) to the β1-β2 region of α-synuclein. To verify the binding of these regions, the dissociation constant (K_{d}) values were measured by MST using synthesized peptide fragments from α-synuclein, αsyn(35-45) (β1 strand) and αsyn(33-58) (β1-β2 region), and SUMO1(15-55). As a result, SUMO1(15-55) only interacted with αsyn(35-45), and it has no binding with αsyn(33-58) (Table 2).

There is no lysine or arginine between residues 60-80 and C-terminus (residues 102-140) in the amino acid sequence of α-synuclein. It means these regions cannot be trypsinized into short fragments and detected by NanoLC-FTICR-MS. Thus, αsyn(74-84), an essential region for α-synuclein filament assembly, and α-synuclein(1-100) were produced to perform MST with SUMO1(15-55). As a result, αsyn (74-84) could bind to SUMO1(15-55), and α-synuclein (1-100) had a similar binding ability compared with full-length α-synuclein (Table 2), which means SUMO1(15-55) does not interact with the C-terminus of α-synuclein.

To explore the potential binding mechanisms, online SUMO interaction motif (SIM) prediction software (Zhao et al., 2014) was adopted to figure out the SIM on α-synuclein. The threshold was set as zero and the result indicated three possible regions, residues 37-41 (P value: 0.202), residues 48-52 (P value: 0.819) and residues 70-74 (P value: 0.643). This is consistent with the facts: αsyn(35-45) showed a strong binding to SUMO1(15-55); SUMO1(15-55) could reduce the cytotoxicity of NACore (residues 68-78); the binding on β2 strand could only be detected by cross-linking but could not be measured by MST. The binding ability between SUMO1(15-55) and another amyloidogenic peptide 42-mer beta-amyloid peptide (Aβ42) was also tested, the dominant Aβ species in the amyloid plaques found in the brain of Alzheimer's patients (Luhrs et al., 2005). Bovine serum albumin (BSA) was used as a negative control. Compared with negative control, the binding ability to Aβ42 was increased about 20-fold (Table 2). Combining all these data, it was hypothesized that the SIM and amyloidogenic structure are two influence factors involved in the binding to SUMO1(15-55).

### DISCUSSION

The findings here report the direct inhibition effect of SUMO1 variants for α-synuclein aggregation for the first time. The full-length protein was truncated into a short therapeutic peptide SUMO1(15-55), which can interact with α-synuclein, abrogate the aggregation, reduce cytotoxicity and ameliorate disease symptoms in Drosophila model of Parkinson's disease. This study has also illustrated the interaction between SUMO1(15-55) and Aβ42, thus providing a possibility to design therapeutic peptides for other neurodegenerative diseases.

Although numbers of peptide-based inhibitors of amyloid fibrils have been proposed (Funke and Willbold, 2012; Sciarretta et al., 2006), this novel therapeutic peptide SUMO1(15-55) possesses unique features and advantages. It is derived from a common human protein existing in human body with no propensity to aggregate, thus it is likely to be less immunogenic than many proteins and less aggregation-prone than β-sheet breaker peptides derived from disease-specific amyloidogenic sequences. In attempting to translate peptide into clinical application, there are hurdles need to be overcome: bioavailability issue, degradation by protease, limit ability to cross cell membrane and blood-brain barrier. These abilities can be given by modifications, for instance, acetylation, PEGylation, addition of a hydrophobic tail or charge tail. In addition, if the atomic structure of SUMO1(15-55)-α-synuclein protein complex is solved, the inhibition effect can be enhanced by backbone modification or further truncation of SUMO1(15-55) into a smaller peptide.

### Example 2

### INTRODUCTION

This study provides insight in the nature of the binding interaction between SUMO(15-55) and α-synuclein. Experiments in Drosophila show that SUMO(31-55) peptide can inhibit α-synuclein induced neurodegeneration in vivo. Moreover, experimental data obtained in this study demonstrate that (1) addition of N-terminal a poly-arginine sequence to the SUMO(31-55) peptide can promote its uptake into cells; and (2) addition of N-terminal His-tag sequence to SUMO(31-55) improves its ability significantly, allowing it to inhibit α-synuclein aggregation at even sub-stochiometric levels.

### MATERIALS AND METHODS

### Thioflavin T Assay

Thioflavin T (ThT) assay was performed as previously described⁵ with minor modifications. Freshly purified 70 μM α-synuclein in the absence and presence of equimolar ratio of SUMO1(15-55)/SUMO1(L44E, L47R), R8-SUMO1 (31-55), or His-SUMO1(31-55), 40 μM Thioflavin T (ThT) (Sigma-Aldrich) and 0.4 mM SDS were dissolved in reaction buffer (20 mM HEPES, 150 mM NaCl, pH 7.4) with a final volume of 150 μL in each well. The potential pre-formed aggregates in α-synuclein were removed by centrifugation at 15,000 rpm for 15 min and filtering through a 0.2 μm filter before the experiments. Reactions were performed in a black clear-bottomed 96-well plate (Nunc, Thermo Fisher Scientific) sealed with clear adhesive sealing sheet (Thermo Fisher Scientific) to avoid evaporation and loaded into an Infinite® M1000 plate reader (Tecan), incubated at 37° C. for 7 days without agitation. The fluorescence was recorded at 485 nm with excitation wavelength of 450 nm at the interval of 30 min.

### Microscale Thermophoresis

Protein targets were labelled using the Alexa Fluor 647 NHS ester dye (Thermo Fisher Scientific) according to the manufacturer's instructions. Unreacted dye was removed with desalting column (2K MWCO, Bio-Rad). The purity was monitored by measuring the ratio of protein to dye after the clean-up procedure. The labelled targets were adjusted to appropriate concentration for detection and kept in ddH2O with 0.1% Tween 20, meanwhile, the ligands were maintained in corresponding buffer (peptides: ddH2O; α-synuclein proteins: 20 mM HEPES, 150 mM NaCl, pH 7.4). For each assay, unlabelled ligand was mixed with equal volume of labelled protein target at 16 different serially diluted concentrations at room temperature. After 10 min incubation, the samples were loaded into standard Monolith NT.115 Capillaries (NanoTemper Technologies) and measured using a Monolith NT.115 instrument (NanoTemper Technologies. Instrument parameters were adjusted to 20% LED power and medium MST power. The dissociation constants (K_{d}) were calculated using MO.Affinity Analysis v.2.2.4 software (NanoTemper Technologies) as mean±s.e.m. from at least three independent experiments with a single site-specific binding model.

### Computational Docking of α-Synuclein Peptides to SUMO1(15-55)

Given the highly disorder conformation of monomeric α-synuclein and no knowledge regarding the binding site between α-synuclein and SUMO1(15-55), a fragment-based high-resolution global docking protocol (PIPER-FlexPepDock³) was adopted to model the interaction. The amino acid sequence of αsyn (35-45) was served as ligand and docked to SUMO1(15-55) or SUMO1(15-55) (L44E, L47R) which are reproduced from the solution structure of human SUMO1 (PDB ID: 2N1V). PIPER-FlexPepDock was run at the webserver (website: piperfpd.furmanlab.cs.huji.ac.il/). The fragment set representing the peptide conformer ensemble was generated using the Rosetta fragment picker and then docked onto the receptor by rigid-body docking using PIPER Fast Fourier transform (FFT) docking algorithm. Then, the flexible full-atom refinement was performed on the coarse PIPER models using Rosetta FlexPepDock Refinement algorithm. The top-ranking refined models were clustered and ranked based on the reweighted score of the best scoring model in each cluster. The top 1 ranked model was selected as prediction. Molecular graphic figures were made using the molecular visualization system Chimera⁶ or PyMOL⁷.

### Drosophila Genetics and Larval Feeding

The following fly strains were adopted in this study: gmr-GAL4 (RRID: BDSC_1104), elav-GAL4 (RRID: BDSC_458), UAS-α-synuclein-A30P (RRID: BDSC_8147), and UAS-α-synuclein-A53T (RRID: BDSC_8148). All strains were obtained from the Bloomington Drosophila Stock Center. To obtain PD model flies, virgin flies carrying the driver gmr-GAL4 or elav-GAL4 on X chromosome were crossed to males from UAS-α-synuclein-A30P/UAS-α-synuclein-A53T stocks. The progeny of these crosses was maintained on standard cornmeal medium supplemented with dry yeast in 25° C. incubator. For peptide treatment, third instar larvae were fed with 2% sucrose solution supplemented with 60 μM, 120 μM or 240 μM of SUMO1(31-55)/SUMO1(15-55) for 2 h respectively and then continued to culture in standard cornmeal medium at 25° C.⁸.

### Pseudopupil Assay

Pseudopupil assay was performed 3-4 days after eclosion as previously described⁹. Images were acquired by a SPOT Insight CCD camera with a 60×oil objective controlled by the SPOT Advanced software (Diagnostic Instruments Inc.). For each condition, at least 100 ommatidia collected from 10 fly eyes were used to calculate the average number of rhabdomeres per ommatidium.

### Climbing Assay

Climbing assay was performed according to previously described¹⁰. Briefly, a group of 10 flies from each condition were placed in individual plastic vial, and gently tapped to the bottom of the vail. The number of flies that could climb a height of 2 cm within 15 s was recorded. Each trial was repeated five times with 1 min recovery periods between each trials. Total 20 (10 males and 10 females) flies from each condition were tested for one genetic cross.

### Confocal Microscopy

SH-SY5Y cells were planted on 35 mm glass bottom confocal dishes and incubated overnight. After co-incubation with Alexa 488 labelled R8-SUMO1(31-55), cells were fixed with 4% paraformaldehyde for 15 min at room temperature at different time points and visualized by Leica TCS SP8 confocal microscope with 63X oil immersion objective.

### Results and Discussions

Site within SUMO1(15-55) Peptide that Binds to α-Synuclein

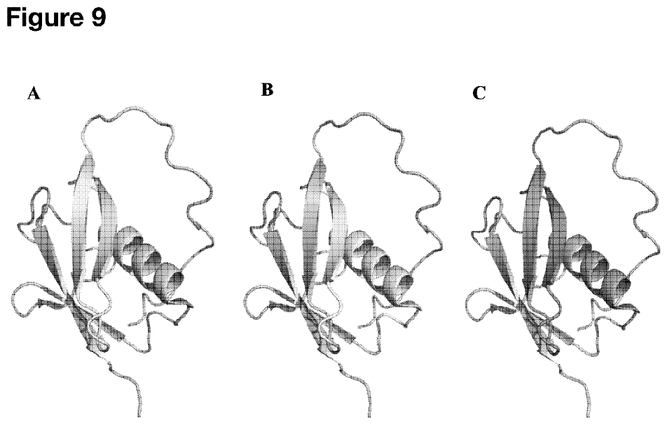

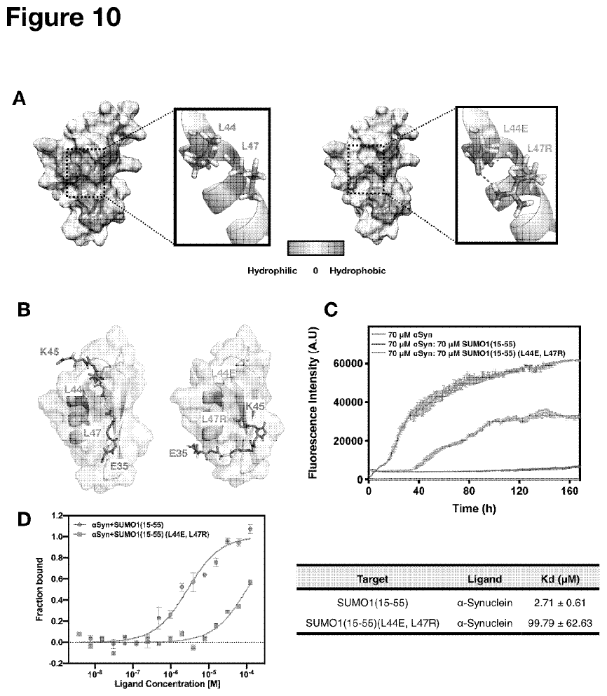

It was hypothesized that SUMO1(15-55) binds to α-synuclein on the putative binding groove formed by the two strands and α-helix via intermolecular hydrophobic interactions. To test this hypothesis, SUMO1(15-55) (L44E, L47R) was designed to validate the hypothesis by mutating the two hydrophobic residues in the putative binding groove into hydrophilic residues to diminish its hydrophobicity, without changing the net charge (FIG. 10a). Computational docking of an α-synuclein peptide (35-45) to SUMO1(15-55) suggested that these mutations should render the SUMO1(15-55) mutant unable to bind to α-synuclein (FIG. 10b). This hypothesis was tested experimentally by determining the inhibitory ability and binding strength between SUMO1(15-55)/SUMO1(L44E, L47R) and α-synuclein. These data show that SUMO1(L44E, L47R) exhibited much weaker inhibition effect at equimolar ratio compared with SUMO1(15-55) (FIG. 10c) and it decreased the binding ability to α-synuclein around 1/40 compared with that of SUMO1(15-55) (FIG. 10d). All these results indicate that the hydrophobic binding groove formed by residues 122, L24, V26, 134, F36, V38, L44, and L47 is the region on SUMO1(15-55) that binds to α-synuclein, thereby suppressing its aggregation.

These data indicates that a SUMO-derived peptide comprised of a β-sheet and α-helix to form a hydrophobic groove that binds to α-synuclein, such as a SUMO1 fragment (e.g., the 15-55 or 31-55 fragment of SEQ ID NO:1) having all or some residues 122, L24, V26, 134, F36, V38, L44, and L47 of SEQ ID NO:1 within the fragment preserved, is useful for inhibiting α-synuclein aggregation and therefore useful for treating neurodegenerative diseases.

Site within α-Synuclein that Binds the SUMO1(15-55)

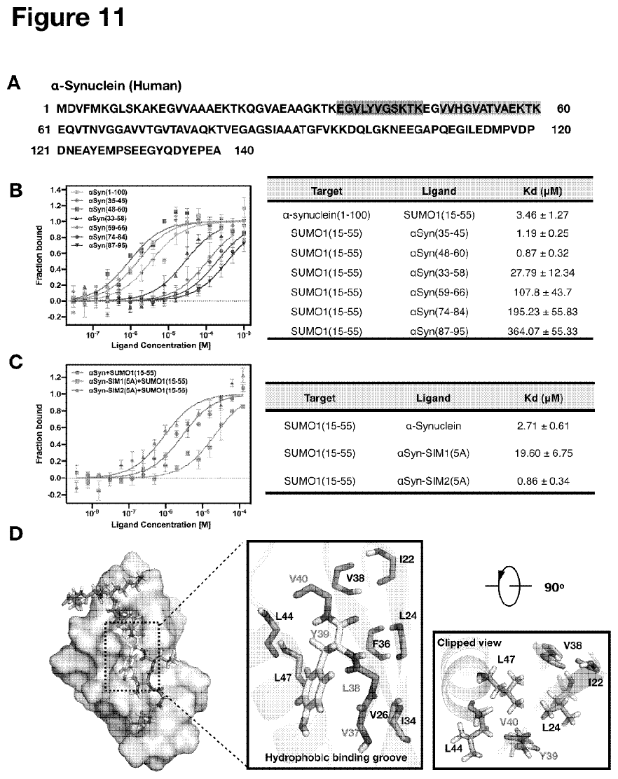

After confirmation of binding site on SUMO1(15-55), the potential interaction sites on α-synuclein was further explored. To validate the interaction region, α-synuclein (1-100) was produced to examine whether the truncation of the C-terminus domain will affect the interaction. It turned out that α-synuclein (1-100) showed comparable K_{d} value with full-length α-synuclein, which means the C-terminus has limited effect on this interaction (FIG. 11b). The affinities between SUMO1(15-55) and β-strand peptides were then evaluated corresponding to the five β-strands in fibrillar α-synuclein designated according to Vilar et al¹. Peptides derived from these strands were synthesized and separately tested by MST to identify potential interaction regions. The β1-strand peptide-αsyn (35-45) with the predicted SIM (³⁷VLYV⁴⁰) and the β2-strand peptide αsyn(48-60) containing a SIM-like motif (⁴⁸VVHGV⁵²) with an extra amino acid in Type β motif ([V/I]-[V/I]-[X]-[V/I/L])² were found to have reasonable affinity to SUMO1(15-55), while the β3, β4, β5-stand peptides (αsyn(59-66), αsyn(74-84), αsyn(87-95)) showed only weak interaction. Moreover, a hairpin construct comprising both β1- and β2-strands—αsyn(33-58) exhibited weaker binding ability, around 1/27 compared with the sole β1, β2-strand peptides (FIG. 11b).

These data suggest that SUMO1(15-55) preferably interacts with fragments without secondary structure, in accordance with the previous results that SUMO1(15-55) showed limited effect on αsyn pre-formed fibrils. Two α-synuclein mutants with quintuple alanine mutations in either of the predicted SIM region (αsyn-SIM1(5A)) and SIM-like motif region (αsyn-SIM2(5A)) were subsequently produced to investigate the sequence selection in this interaction. As shown in FIG. 11c, the quintuple alanine mutation in SIM1 reduced the binding affinity to about 1/7, while the mutation in SIM2 did not affect the interaction but instead enhanced the binding ability, which suggests the amino acid sequence in SIM affected the binding with SUMO1(15-55) and the SIM-like motif competed with SIM for the recognition to SUMO1(15-55). A SUMO1(15-55)-αsyn(35-45) complex model was produced by computational prediction using a fragment-based high-resolution global docking protocol (PIPER-FlexPepDock³). In agreement with the hypothesis, αsyn (35-45) binds to the putative hydrophobic binding groove on SUMO1(15-55) via SIM (³⁷VLYV⁴⁰) (FIG. 11d).

With previous experiments narrowing down the binding region on α-synuclein to the two SUMO interaction motifs (SIM), SIM 1 (residues 35-45) and SIM 2 (residues 48-60), recently performed further experiments were set to validate the binding sites on α-synuclein by producing α-synuclein mutants that can potentially block the interaction.

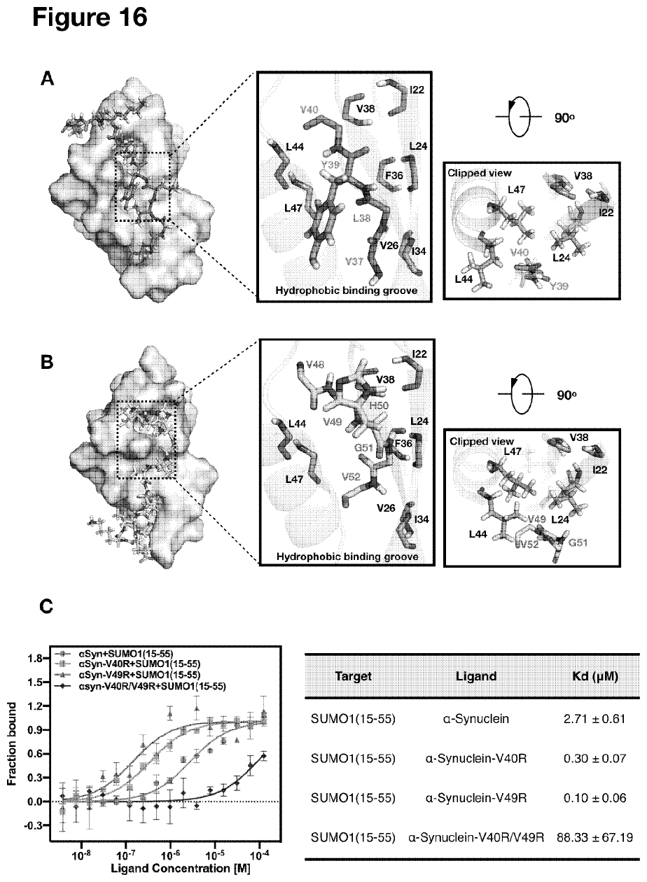

In the predicted SUMO1(15-55)-αsyn peptide models, as expected, the SIMs (³⁷VLYV⁴⁰ and ⁴⁸VVHGV⁵²) on α-synuclein bind to the putative hydrophobic binding groove on SUMO1(15-55) (FIG. 3a, b). Based on the hypothesis, the interactions between α-synuclein and SUMO1(15-55) depend on the SIM recognition and intermolecular hydrophobic interactions, thus, α-synuclein-(V40R), α-synuclein-(V49R), and α-synuclein-(V40R,V49R) mutants were designed, which should weaken the hydrophobicity of these regions and thereby inhibit SUMO(15-55) binding to one or both SIM sites. Notably, mutation on either one SIM did not abolish the SUMO(15-55)-α-synuclein interaction, but the interaction was inhibited by mutations on both sites simultaneously (FIG. 16c).

These data provides further support to the notion that SUMO1(15-55) binds to α-synuclein via its SIM1 (³⁷VLYV⁴⁰) and SIM 2 (⁴⁸VVHGV⁵²) motifs, which in turn hinders formation higher order toxic species. It should be noted that the importance of these sites for inhibiting α-synuclein aggregation has been highlighted recently by Doherty et al.¹¹.

Thus, α-synuclein fragments (i.e., less than full length α-synuclein, for example, no more than 50, 40, 30, or 20 amino acids in total length), which are polypeptides comprising the segment of 35-45 or 48-60 of SEQ ID NO:7, such as the 37-41, or 37-40, or 48-52 segment of SEQ ID NO:7, optionally with one or more heterologous peptide sequences (for example, each no more than 6, 8, 10, 12, 15, or 20 amino acids in length, such as a poly-His or poly-Arg tag) added at the N- and/or C-terminus, may be used in a manner substantially the same as the SUMO-derived peptides described herein for essentially the same purposes, e.g., for suppressing α-synuclein aggregation in cells and for treating neurodegenerative diseases such as Parkinson's Disease. Similarly, peptides derived from α-synuclein comprising the full length α-synuclein or a fragment of α-synuclein as described herein including one or more mutation in SIM1 or SIM2, preferably not in both (such as substitution mutant V40R or V49R but not double mutant V40R/V49R), optionally with one or more heterologous peptide sequences (for example, each no more than 6, 8, 10, 12, 15, or 20 amino acids in length, such as a poly-His or poly-Arg tag) added at the N- and/or C-terminus, may be used in a manner substantially the same as the SUMO-derived peptides described herein for essentially the same purposes, e.g., for suppressing α-synuclein aggregation in cells and for treating neurodegenerative diseases such as Parkinson's Disease. Also provided are nucleic acids encoding such α-synuclein fragments or peptides derived from α-synuclein, expression cassettes directing the expression of the α-synuclein fragments or peptides derived from α-synuclein, vectors comprising the expression cassettes, or host cells comprising the nucleic acids, expression cassettes, or vectors, as well as a composition comprising a physiologically acceptable excipient and an effective amount of one such α-synuclein fragment or peptide derived from α-synuclein.

### The Neuroprotective Effect of SUMO1(15-55) Peptide in α-Synuclein Transgenic Drosophila

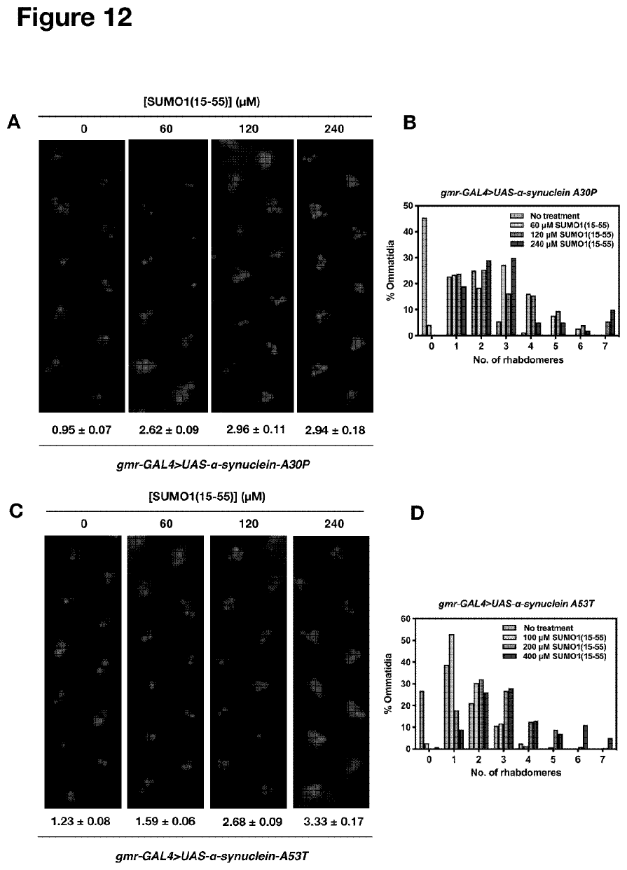

The neuroprotective effect of SUMO1(15-55) was previously shown in wild-type α-synuclein transgenic Drosophila. In this study, new data were generated to show the neuroprotective effect of SUMO1(15-55) in α-synuclein transgenic Drosophila expressing α-synuclein mutants (α-synuclein A30P and α-synuclein A53T) that is related to familial Parkinson's disease. Pseudopupil assay quantitates the neurodegeneration by counting the rhabdomeres in each ommatidium⁴. In contrast to no treatment control group, SUMO1(15-55) treatment groups significantly ameliorated the neurodegeneration process induced by α-synuclein-A30P (FIG. 12a, b) or α-synuclein-A53T (FIG. 12c, d) in a dose-dependent manner.

These data indicate that SUMO1(15-55) is useful for treating familial Parkinson's disease caused by distinct mutations in α-synuclein other than the overexpression of wild-type α-synuclein.

### The Neuroprotective Effect of SUMO1(31-55) Peptide in α-Synuclein Transgenic Drosophila

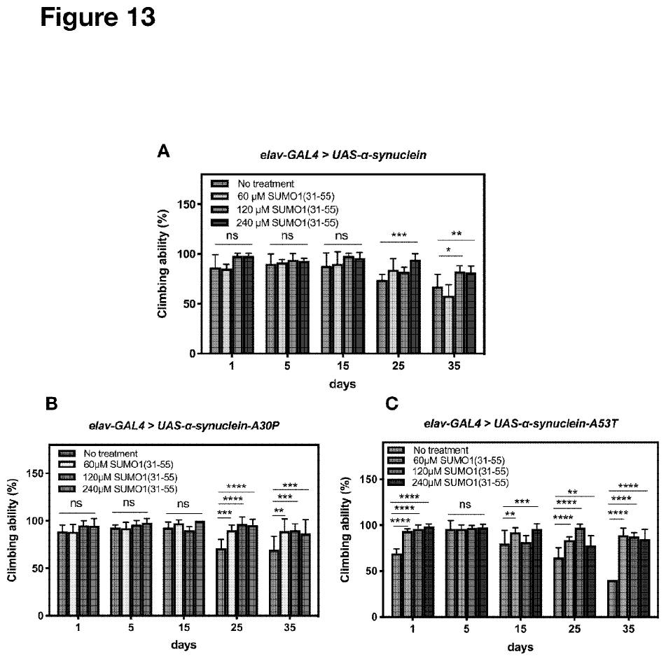

It was previously disclosed that a shorter peptide SUMO1(31-55) can also inhibit α-synuclein aggregation in vitro. The neuroprotective effect of SUMO1(31-55) was tested in α-synuclein (wild-type, A30P, A53T) transgenic Drosophila with climbing assay. The shorter peptide SUMO1(31-55) improved the locomotor dysfunction in the pilot behavioral studies in all three α-synuclein transgenic Drosophila strains (FIG. 13).

These data indicate that SUMO1(31-55) possesses comparable protective ability for α-synuclein induced neurodegeneration in vivo.

### Cell Penetrating Function of R8-SUMO1(31-55)

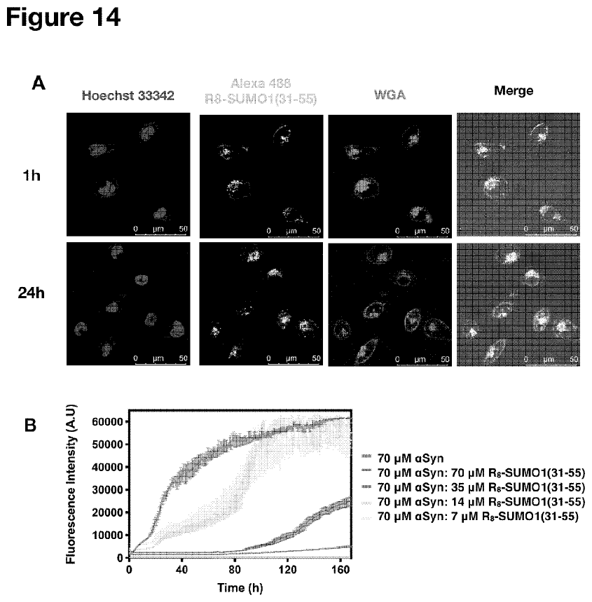

Previous behavioral tests have shown the protective effects of SUMO1(31-55) in α-synuclein transgenic Drosophila models. To further investigate the therapeutic effects in mouse model, a poly-arginine tag was added in the N-terminus of SUMO1(31-55) to enhance its cell penetrating function and the potential therapeutic effects. As shown in FIG. 14a, the presence of the poly-arginine tag led to SUMO1(31-55) internalization into SH-SY5Y cells at a high efficiency in a time course manner, while the poly-arginine tag did not impact the inhibitory ability of SUMO1(31-55) for α-synuclein aggregation in vitro (FIG. 14b).

These data indicate that R8-SUMO1(31-55) can serve as a peptide drug with improved properties in the treatment of neurodegenerative diseases.

### His-SUMO1(31-55) Sub-Stoichiometrically Inhibits α-Synuclein Aggregation In Vitro

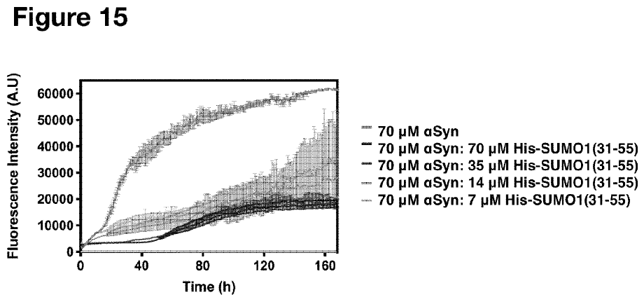

It was also found that incorporation of an N-terminal 10×His tag improved the inhibitory effect of SUMO1(31-55) significantly, allowing it to inhibit α-synuclein aggregation at sub-stoichiometric levels. This is a major advance, since previous constructs lose their ability to inhibit α-synuclein at a ratio of 1:5 or 1:10 α-synuclein: SUMO peptide inhibitor. However, the His-tagged SUMO1(31-55) exhibited partial inhibition of α-synuclein aggregation in vitro even at these reduced levels (FIG. 15).

These data indicate that His-SUMO1(31-55) is an even more potent peptide drug for the treatment of synucleinopathies.

While SUMO2 only shares around 50% sequence identity with SUMO1, it shows structural homology to SUMO1 and has also been shown to interact with α-synuclein. Thus, the inhibition effect of SUMO2(16-88) and SUMO2(16-51) was further investigated. These fragments are in alignment with the SUMO1(15-92) and SUMO1(15-55) peptides studied previously, albeit without a 5-amino acid N-terminal tail present in these SUMO1 peptides, due to their being absent in the SUMO2 sequence.

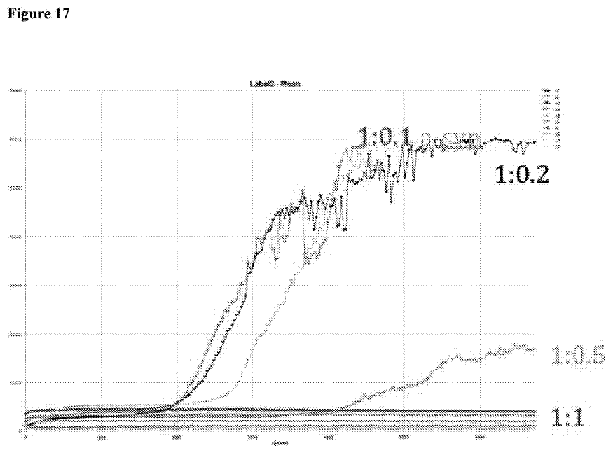

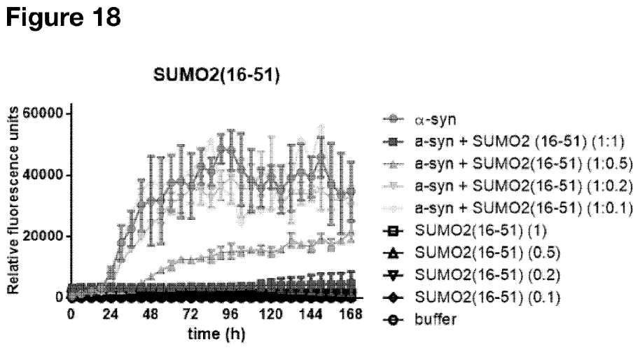

As observed for SUMO1(15-92), SUMO2(16-88) completely inhibited α-synuclein aggregation at an equimolar ration to α-synuclein, while it delayed aggregation at a half molar ratio (FIG. 17). The shorter SUMO2 peptide, SUMO2(16-51), also suppressed α-synuclein aggregation at an equimolar ratio, while it delayed the initiation of the aggregation for 2 days at a half molar ratio (FIG. 18). These data indicate that SUMO2 fragments also possess the ability to inhibit α-synuclein aggregation comparable to their SUMO1 counterparts.

All patents, patent applications, and other publications, including GenBank Accession Numbers, cited in this application are incorporated by reference in the entirety for all purposes.

SEQUENCE LISTING

SEQ ID NO: 1 Human SUMO1 protein amino acid
sequence, GenBank Accession No. AAC50996.1
MSDQEAKPSIEDLGDKKEGEYIKLKVIGQDSSEIHFKVKMTTHLKKLKES
YCQRQGVPMNSLRFLFEGQRIADNHTPKELGMEEEDVIEVYQEQTGGHST
V
SEQ ID NO: 2 Human SUMO1 coding sequence, GenBank
Accession No. NG_011679.1
ATGTCTGACCAGGAGGCAAAACCTTCAACTGAGGACTTGGGGGATAAGAA
GGAAGGTGAATATATTAAACTCAAAGTCATTGGACAGGATAGCAGTGAGA
TTCACTTCAAAGTGAAAATGACAACACATCTCAAGAAACTCAAAGAATCA
TACTGTCAAAGACAGGGTGTTCCAATGAATTCACTCAGGTTTCTCTTTGA
GGGTCAGAGAATTGCTGATAATCATACTCCAAAAGAACTGGGAATGGAGG
AAGAAGATGTGATTGAAGTTTATCAGGAACAAACGGGGGGTCATTCAACA
GTT
SEQ ID NO: 3 Human SUMO2 protein amino acid
sequence, GenBank No. AAH71645.1
MADEKPKEGVKTENDDHINLKVAGQDGSVVQFKIKRHTPLSKLMKAYCER
QGLSMRQIRFRFDGQPINETDTPAQLEMEDEDTIDVFQQQTGGVY
SEQ ID NO: 4 Human SUMO3 protein amino acid
sequence, NCBI Reference Sequence: NP_008867.2
MSEEKPKEGVKTENDHINLKVAGQDGSVVQFKIKRHTPLSKLMKAYCERQ
GLSMRQIRFRFDGQPINETDTPAQLEMEDEDTIDVFQQQTGGVPESSLAG
HSF
SEQ ID NO: 5 Human SUMO4 protein amino acid
sequence, NCBI Reference Sequence: NP_001002255.1
MANEKPTEEVKTENNNHINLKVAGQDGSVVQFKIKRQTPLSKLMKAYCEP
RGLSVKQIRFRFGGQPISGTDKPAQLEMEDEDTIDVFQQPTGGVY
SEQ ID NO: 6 Human SUMO protein amino acid
sequence
MSDLEAKPSTEHLGDKIKDEDIKLRVIGQDSSEIHFKVKMTTPLKKLKKS
YCQRQGVPVNSLRFLFEGQRIADNHTPEELGMEEEDVIEVYQEQIGGHST
V
SEQ ID NO: 7 Human α-synuclein protein amino acid
sequence, NCBI Reference Sequence: NP_000336.1
(The predicted SIM motif in α-synuclein
(residues 37-41) is underlined)
MDVFMKGLSKAKEGVVAAAEKTKQGVAEAAGKTKEGVLYVGSKTKEGVVH
GVATVAEKTKEQVTNVGGAVVTGVTAVAQKTVEGAGSIAAATGFVKKDQL
GKNEEGAPQEGILEDMPVDPDNEAYEMPSEEGYQDYEPEA

TABLE 1
Theoretical and experimental masses of digested α-synuclein-SUMO1 dipeptides
containing the sulfo-GMBS cross-linker

| | | | Experimental | |
|---|---|---|---|---|
| | | | monoisotopic | |
| | | Theoretical | mass [M + H]⁺ | |
| Cross-linked dipeptides | Sequence | mass (Da) | (Da) | Score |
| 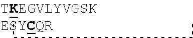 | αsyn(33-43) + SUMO1(49-54) | 2131.36 | 2130.017 | 72 |
| 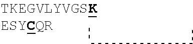 | αsyn(33-43) + SUMO1(49-54) | 2131.36 | 2130.017 | 66 |
| 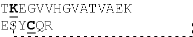 | αsyn(44-58) + SUMO1(49-54) | 2475.73 | 2474.198 | 67 |
| 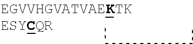 | αsyn(46-60) + SUMO1(49-54) | 2475.73 | 2474.198 | 41 |
| 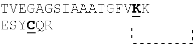 | αsyn(81-97) + SUMO1(49-54) | 2557.83 | 2556.240 | 75 |

TABLE 2
The dissociation constants of interactions measured
by microscale thermophoresis

| Target | Ligand | Kd (μM) |
|---|---|---|
| NHS-647 α-synuclein | SUMO1(15-55) | 4.71 ± 1.42 |
| NHS-647 WT α-synuclein | SUMO1(15-55) | 2.73 ± 1.11 |
| (1-100) | | |
| NHS-647 SUMO1(15-55) | αsyn(35-45) | 1.15 ± 0.54 |
| NHS-647 SUMO1(15-55) | αsyn(33-58) | No binding was detected |
| NHS-647 SUMO1(15-55) | αsyn(74-84) | 2.21 ± 0.71 |
| NHS-647 SUMO1(15-55) | BSA | 64.71 ± 24.82 |
| NHS-647 SUMO1(15-55) | Aβ42 | 2.94 ± 0.69 |

TABLE 3
Primers used in this study

| Primer name | Sequence |
|---|---|
| pET-α-synuclein (Fwrd) | 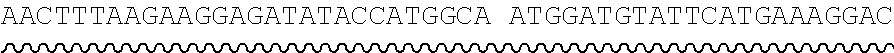 |
| pET-α-synuclein (Rev) | CAGTGGTGGTGGTGGTGGTGCTCGAGTTAGGCTTCAGGTTCGTAGTC |
| pET-α-synuclein (1-100) (Rev) | CAGTGGTGGTGGTGGTGGTGCTCGAGTTACAACTGGTCCTTTTTGACAAAG |
| pET28a-SUMO1(1-97) (Fwd) | GCCGCGCGGCAGCCATATGTCTGACCAGGAGGCAA |
| pET28a-SUMO1(1-97) (Rev) | GTGGTGGTGGTGCTCGAGTTA ACCCCCCGTTTGTTCCTGATA |
| pET28a-SUMO1(15-92) (Fwrd) | GCCGCGCGGCAGCCATATGGATAAGAAGGAAGGTGAATA |
| pET28a-SUMO1(15-92) (Rev) | GTGGTGGTGGTGCTCGAGTTACTGATAAACTTCAATCACATCT |
| pBFC-SUMO1(15-55)-VC155 (Fwrd) | TGGAGGCCCGAATTCGGTCGACATGGATAAGAAGGAAGGTGA |
| pBiFC-SUMO1(15-55)-VC155 (Rev) | TTGCACGCCGGACGGGTACCCTGTCTTTGACAGTATGATT |
| pBiFC-α-synuclein-VN173 (Fwrd) | AAAGACGATGACGACAAGCTTATGGATGTATTCATGAAAGGA |
| pBiFC-α-synuclein-VN173 (Rev) | GATGGATCTTCTAGAGTCGACGGCTTCAGGTTCGTAGTC |
| pBiFC-VN173-α-synclein (modify | CTGATATCGGTACCAGTCGACTCTAGAAGATCCAT |
| backbone) (Fwrd) | |
| pBiFC-VN173-α-synclein (modify | CAGGGATGCCACCCGGGATCCTGCCTCGATGTTGTGGC |
| backbone) (Rev) | |
| pBiFC-VN173-α-synclein (Fwd) | ACAACATCGAGGCAGGATCCATGGATGTATTCATGAAAGGAC |
| pBiFC-VN173-α-synclein (Rev) | 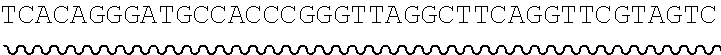 |

### REFERENCES FOR EXAMPLE 1

Angot, E., Steiner, J. A., Lema Tome, C. M., Ekstrom, P., Mattsson, B., Bjorklund, A., and Brundin, P. (2012). Alpha-synuclein cell-to-cell transfer and seeding in grafted dopaminergic neurons in vivo. PLoS One 7, e39465.
Appel-Cresswell, S., Vilarino-Guell, C., Encarnacion, M., Sherman, H., Yu, I., Shah, B., Weir, D.,
Thompson, C., Szu-Tu, C., Trinh, J., et al. (2013). Alpha-synuclein p.H50Q, a novel pathogenic mutation for Parkinson's disease. Mov Disord 28, 811-813.
Aulic, S., Le, T. T. N., Moda, F., Abounit, S., Corvaglia, S., Casalis, L., Gustincich, S., Zurzolo, C., Tagliavini, F., and Legname, G. (2014). Defined alpha-synuclein prion-like molecular assemblies spreading in cell culture. Bmc Neurosci 15.
Auluck, P. K., and Bonini, N. M. (2002). Pharmacological prevention of Parkinson disease in Drosophila. Nat Med 8, 1185-1186.
Auluck, P. K., Meulener, M. C., and Bonini, N. M. (2005). Mechanisms of Suppression of {alpha}-Synuclein Neurotoxicity by Geldanamycin in Drosophila. J Biol Chem 280, 2873-2878. Berger, Z., Ttofi, E. K., Michel, C. H., Pasco, M. Y., Tenant, S., Rubinsztein, D. C., and O'Kane,
C. J. (2005). Lithium rescues toxicity of aggregate-prone proteins in Drosophila by perturbing Wnt pathway. Hum Mol Genet 14, 3003-3011.
Chau, K. W, Chan, W. Y., Shaw, P. C., and Chan, H. Y. (2006). Biochemical investigation of Tau protein phosphorylation status and its solubility properties in Drosophila. Biochem Biophys Res Commun 346, 150-159.
Chen, X., de Silva, H. A., Pettenati, M. J., Rao, P. N., St George-Hyslop, P, Roses, A. D., Xia, Y, Horsburgh, K., Ueda, K., and Saitoh, T. (1995). The human NACP/alpha-synuclein gene: chromosome assignment to 4q21.3-q22 and TaqI RFLP analysis. Genomics 26, 425-427. Crowther, R. A., Jakes, R., Spillantini, M G., and Goedert, M. (1998). Synthetic filaments assembled from C-terminally truncated alpha-synuclein. FEBS Lett 436, 309-312.
Desplats, P., Lee, H. J., Bae, E. J., Patrick, C., Rockenstein, E., Crews, L., Spencer, B., Masliah, E., and Lee, S. J. (2009). Inclusion formation and neuronal cell death through neuron-to-neuron transmission of alpha-synuclein. Proc Natl Acad Sci USA 106, 13010-13015.
Feany, M. B., and Bender, W. W. (2000). A Drosophila model of Parkinson's disease. Nature 404, 394-398.
Funke, S. A., and Willbold, D. (2012). Peptides for therapy and diagnosis of Alzheimer's disease. Curr Pharm Des 18, 755-767.
Geiss-Friedlander, R., and Melchior, F. (2007). Concepts in sumoylation: a decade on. Nat Rev Mol Cell Biol 8, 947-956.
Giasson, B. I., Murray, I. V. J., Trojanowski, J. Q., and Lee, V. M. Y. (2001). A hydrophobic stretch of 12 amino acid residues in the middle of alpha-synuclein is essential for filament assembly. Journal of Biological Chemistry 276, 2380-2386.
Giasson, B. I., Uryu, K., Trojanowski, J. Q., and Lee, V. M. (1999). Mutant and wild type human alpha-synucleins assemble into elongated filaments with distinct morphologies in vitro. J Biol Chem 274, 7619-7622.
Giehm, L., Oliveira, C. L., Christiansen, G., Pedersen, J. S., and Otzen, D. E. (2010). SDS-induced fibrillation of alpha-synuclein: an alternative fibrillation pathway. J Mol Biol 401, 115-133. Groenning, M. (2010). Binding mode of Thioflavin T and other molecular probes in the context of amyloid fibrils-current status. J Chem Biol 3, 1-18.
Guerrero-Ferreira, R., Taylor, N. M., Mona, D., Ringler, P, Lauer, M. E., Riek, R., Britschgi, M., and Stahlberg, H. (2018). Cryo-EM structure of alpha-synuclein fibrils. Elife 7. Hansen, C., Angot, E., Bergstrom, A. L., Steiner, J. A., Pieri, L., Paul, G., Outeiro, T. F., Melki, R., Kallunki, P., Fog, K., et al. (2011). alpha-Synuclein propagates from mouse brain to grafted dopaminergic neurons and seeds aggregation in cultured human cells. J Clin Invest 121, 715-725. Hecker, C. M., Rabiller, M., Haglund, K., Bayer, P., and Dikic, I. (2006). Specification of SUMO1- and SUMO2-interacting motifs. Journal of Biological Chemistry 281, 16117-16127. Janer, A., Werner, A., Takahashi-Fujigasaki, J., Daret, A., Fujigasaki, H., Takada, K., Duyckaerts, C., Brice, A., Dejean, A., and Sittler, A. (2010). SUMOylation attenuates the aggregation propensity and cellular toxicity of the polyglutamine expanded ataxin-7. Hum Mol Genet 19, 181-195.
Kalia, L. V., and Lang, A. E. (2015). Parkinson's disease. Lancet 386, 896-912.
Kruger, R., Kuhn, W, Muller, T., Woitalla, D., Graeber, M., Kosel, S., Przuntek, H., Epplen, J. T., Schols, L., and Riess, 0. (1998). Ala30Pro mutation in the gene encoding alpha-synuclein in Parkinson's disease. Nat Genet 18, 106-108.
Krumova, P., Meulmeester, E., Garrido, M., Tirard, M., Hsiao, H. H., Bossis, G., Urlaub, H., Zweckstetter, M., Kugler, S., Melchior, F., et al. (2011). Sumoylation inhibits alpha-synuclein aggregation and toxicity. J Cell Biol 194, 49-60.
Krumova, P., and Weishaupt, J. H. (2013). Sumoylation in neurodegenerative diseases. Cell Mol Life Sci 70, 2123-2138.
Lesage, S., Anheim, M., Letournel, F., Bousset, L., Honore, A., Rozas, N., Pieri, L., Madiona, K., Durr, A., Melki, R., et al. (2013). G51D alpha-synuclein mutation causes a novel parkinsonian-pyramidal syndrome. Ann Neurol 73, 459-471.
Loriol, C., Parisot, J., Poupon, G., Gwizdek, C., and Martin, S. (2012). Developmental regulation and spatiotemporal redistribution of the sumoylation machinery in the rat central nervous system. PLoS One 7, e33757.
Luhrs, T., Ritter, C., Adrian, M., Riek-Loher, D., Bohrmann, B., Doeli, H., Schubert, D., and Riek, R. (2005). 3D structure of Alzheimer's amyloid-beta(1-42) fibrils. P Natl Acad Sci USA 102, 17342-17347.
Luk, K. C., Song, C., O'Brien, P., Stieber, A., Branch, J. R., Brunden, K. R., Trojanowski, J. Q., and Lee, V. M. Y. (2009). Exogenous alpha-synuclein fibrils seed the formation of Lewy body-like intracellular inclusions in cultured cells. P Natl Acad Sci USA 106, 20051-20056.
Marsh, J. L., Pallos, J., and Thompson, L. M. (2003). Fly models of Huntington's disease. Hum Mol Genet 12 Spec No 2, R187-193.
Martin, S., Nishimune, A., Mellor, J. R., and Henley, J. M. (2007). SUMOylation regulates kainate-receptor-mediated synaptic transmission. Nature 447, 321-325.
Mason, J. M., and Fairlie, D. P. (2015). Toward peptide-based inhibitors as therapies for Parkinson's disease. Future Med Chem 7, 2103-2105.
Minty, A., Dumont, X., Kaghad, M., and Caput, D. (2000). Covalent modification of p73 alpha by SUMO-1—Two-hybrid screening with p73 identifies novel SUMO-1-interacting proteins and a SUMO-1 interaction motif. Journal of Biological Chemistry 275, 36316-36323.
Morell, M., Espargaro, A., Aviles, F. X., and Ventura, S. (2008). Study and selection of in vivo protein interactions by coupling bimolecular fluorescence complementation and flow cytometry. Nat Protoc 3, 22-33.
Mukherjee, S., Thomas, M., Dadgar, N., Lieberman, A. P., and Iniguez-Lluhi, J. A. (2009). Small ubiquitin-like modifier (SUMO) modification of the androgen receptor attenuates polyglutamine-mediated aggregation. J Biol Chem 284, 21296-21306.
Outeiro, T. F., Kontopoulos, E., Altmann, S. M., Kufareva, I., Strathearn, K. E., Amore, A. M., Volk, C. B., Maxwell, M. M., Rochet, J. C., McLean, P. J., et al. (2007). Sirtuin 2 inhibitors rescue alpha-synuclein-mediated toxicity in models of Parkinson's disease. Science 317, 516-519.
Park, S. M., Jung, H. Y, Chung, K. C., Rhim, H., Park, J. H., and Kim, J. (2002). Stress-induced aggregation profiles of GST-alpha-synuclein fusion proteins: role of the C-terminal acidic tail of alpha-synuclein in protein thermosolubility and stability. Biochemistry 41, 4137-4146. Pasanen, P., Myllykangas, L., Siitonen, M., Raunio, A., Kaakkola, S., Lyytinen, J., Tienari, P. J., Poyhonen, M., and Paetau, A. (2014). Novel alpha-synuclein mutation A53E associated with atypical multiple system atrophy and Parkinson's disease-type pathology. Neurobiol Aging 35, 2180 e2181-2185.
Plant, L. D., Dowdell, E. J., Dementieva, I. S., Marks, J. D., and Goldstein, S. A. (2011). SUMO modification of cell surface Kv2.1 potassium channels regulates the activity of rat hippocampal neurons. J Gen Physiol 137, 441-454.
Proukakis, C., Dudzik, C. G., Brier, T., MacKay, D. S., Cooper, J. M., Millhauser, G. L., Houlden, H., and Schapira, A. H. (2013). A novel alpha-synuclein missense mutation in Parkinson disease. Neurology 80, 1062-1064.
Rodriguez, J. A., Ivanova, M. I., Sawaya, M. R., Cascio, D., Reyes, F. E., Shi, D., Sangwan, S., Guenther, E. L., Johnson, L. M., Zhang, M., et al. (2015). Structure of the toxic core of alpha-synuclein from invisible crystals. Nature 525, 486-+.
Sciarretta, K. L., Gordon, D. J., and Meredith, S. C. (2006). Peptide-based inhibitors of amyloid assembly. Methods Enzymol 413, 273-312.
Shyu, Y. J., Suarez, C. D., and Hu, C. D. (2008). Visualization of ternary complexes in living cells by using a BiFC-based FRET assay. Nat Protoc 3, 1693-1702.
Souza, J. M., Giasson, B. I., Lee, V. M., and Ischiropoulos, H. (2000). Chaperone-like activity of synucleins. FEBS Lett 474, 116-119.
Vilar, M., Chou, H. T., Luhrs, T., Maji, S. K., Riek-Loher, D., Verel, R., Manning, G., Stahlberg, H., and Riek, R. (2008). The fold of alpha-synuclein fibrils. Proc Natl Acad Sci USA 105, 8637-8642.
Wong, Y C., and Krainc, D. (2017). alpha-synuclein toxicity in neurodegeneration: mechanism and therapeutic strategies. Nat Med 23, 1-13.
Zarranz, J. J., Alegre, J., Gomez-Esteban, J. C., Lezcano, E., Ros, R., Ampuero, I., Vidal, L., Hoenicka, J., Rodriguez, O., Atares, B., et al. (2004). The new mutation, E46K, of alpha-synuclein causes Parkinson and Lewy body dementia. Ann Neurol 55, 164-173.
Zhao, Q., Xie, Y. B., Zheng, Y. Y., Jiang, S., Liu, W. Z., Mu, W. P., Liu, Z. X., Zhao, Y, Xue, Y, and Ren, J. (2014). GPS-SUMO: a tool for the prediction of sumoylation sites and SUMO-interaction motifs. Nucleic Acids Res 42, W325-W330.

### REFERENCES FOR EXAMPLE 2

1 Vilar, M. et al. The fold of alpha-synuclein fibrils. Proc Natl Acad Sci USA 105, 8637-8642, (2008).
2 Song, J., Durrin, L. K., Wilkinson, T. A., Krontiris, T. G. & Chen, Y Identification of a SUMO-binding motif that recognizes SUMO-modified proteins. Proceedings of the National Academy of Sciences 101, 14373-14378, (2004).
3 Alam, N. et al. High-resolution global peptide-protein docking using fragments-based PIPER-FlexPepDock. Plos Comput Biol 13, (2017).
4 Marsh, J. L., Pallos, J. & Thompson, L. M. Fly models of Huntington's disease. Hum Mol Genet 12 Spec No 2, R187-193, (2003).
5 Giehm, L. & Otzen, D. E. Strategies to increase the reproducibility of protein fibrillization in plate reader assays. Anal Biochem 400, 270-281, (2010).
6 Pettersen, E. F. et al. UCSF chimera—A visualization system for exploratory research and analysis. J Comput Chem 25, 1605-1612, (2004).
7 DeLano, W. L. The PyMOL Molecular Graphics System. DeLano Scientific, San Carlos, Calif., USA, (2002).
8 Chau, K. W, Chan, W. Y, Shaw, P. C. & Chan, H. Y. Biochemical investigation of Tau protein phosphorylation status and its solubility properties in Drosophila. Biochem Biophys Res Commun 346, 150-159, (2006).
9 Berger, Z. et al. Lithium rescues toxicity of aggregate-prone proteins in Drosophila by perturbing Wnt pathway. Hum Mol Genet 14, 3003-3011, (2005).
10 Feany, M. B. & Bender, W. W. A Drosophila model of Parkinson's disease. Nature 404, 394-398, (2000).
11 Doherty, C. P. A. et al. A short motif in the N-terminal region of α-synuclein is critical for both aggregation and function. Nature Structural & Molecular Biology 27, 249-259, (2020).

What is claimed is:

1. A SUMO-derived polypeptide comprising a core sequence, which is the 31-55 segment of SEQ ID NO:1, the 27-51 segment of SEQ ID NO:3, or the 26-50 segment of SEQ ID NO:4, or the 27-51 segment of SEQ ID NO:5, or the 31-55 segment of SEQ ID NO:6, wherein the core sequence is conjugated to a heterologous moiety, and wherein the polypeptide suppresses α-synuclein aggregation.

2. The polypeptide of claim 1, wherein the heterologous moiety is a heterologous amino acid sequence.

3. The polypeptide of claim 1, wherein the heterologous moiety is a detectable label.

4. The polypeptide of claim 1, wherein the heterologous moiety is an affinity tag.

5. The polypeptide of claim 1, consisting of the core sequence and one or more heterologous amino acid sequences at the N- and/or C-terminus of the core sequence.

6. The polypeptide of claim 5, consisting of the core sequence of the 15-55, 31-55, 20-92, or 20-55 segment of SEQ ID NO:1, the 10-51, 27-51, 16-88, or 16-51 segment of SEQ ID NO:3, or the 10-50, 26-50, 15-87, or 15-50 segment of SEQ ID NO:4, or the 10-51, 27-51, 16-88, or 16-51 segment of SEQ ID NO:5, or the 15-55, 31-55, 20-92, or 20-55 segment of SEQ ID NO:6 and a poly-arginine or poly-histidine tag at the N-terminus of the core sequence.

7. The polypeptide of claim 6, consisting of the core sequence of the 31-55 segment of SEQ ID NO:1, the 27-51 segment of SEQ ID NO:3, the 26-50 segment of SEQ ID NO:4, the 27-51 segment of SEQ ID NO:5, or the 31-55 segment of SEQ ID NO:6 and a poly-arginine or poly-histidine tag at the N-terminus of the core sequence.

8. A nucleic acid comprising a polynucleotide sequence encoding a core sequence, which is the 31-55 segment of SEQ ID NO:1, the 27-51 segment of SEQ ID NO:3, or the 26-50 segment of SEQ ID NO:4, or the 27-51 segment of SEQ ID NO:5, or the 31-55 segment of SEQ ID NO:6, wherein the nucleic acid further comprises at least one coding sequence for at least one heterologous amino acid sequence at the N- and/or C-terminus of the core sequence.

9. The nucleic acid of claim 8, encoding a fusion protein consisting of the core sequence and one or more heterologous amino acid sequences at the N- and/or C-terminus of the core sequence.

10. The nucleic acid of claim 9, wherein the fusion protein consists of the core sequence of the 15-55, 31-55, 20-92, or 20-55 segment of SEQ ID NO:1, the 10-51, 27-51, 16-88, or 16-51 segment of SEQ ID NO:3, or the 10-50, 26-50, 15-87, or 15-50 segment of SEQ ID NO:4, or the 10-51, 27-51, 16-88, or 16-51 segment of SEQ ID NO:5, or the 15-55, 31-55, 20-92, or 20-55 segment of SEQ ID NO:6 and a poly-arginine or poly-histidine tag at the N-terminus of the core sequence.

11. The nucleic acid of claim 10, wherein the fusion protein consists of the core sequence of the 31-55 segment of SEQ ID NO:1, the 27-51 segment of SEQ ID NO:3, the 26-50 segment of SEQ ID NO:4, the 27-51 segment of SEQ ID NO:5, or the 31-55 segment of SEQ ID NO:6 and a poly-arginine or poly-histidine tag at the N-terminus of the core sequence.

12. An expression cassette comprising a polynucleotide sequence encoding a polypeptide of claim 1 operably linked to a heterologous promoter.

13. The expression cassette of claim 12, wherein the polypeptide consisting of the core sequence and one or more heterologous amino acid sequences at the N- and/or C-terminus of the core sequence.

14. A vector comprising the expression cassette of claim 12 or 13.

15. A host cell comprising the vector of claim 14.

16. A composition comprising a physiologically acceptable excipient and an effective amount of (1) a polypeptide comprising the 31-55 segment of SEQ ID NO:1, the 27-51 segment of SEQ ID NO:3, or the 26-50 segment of SEQ ID NO:4, or the 27-51 segment of SEQ ID NO:5, or the 31-55 segment of SEQ ID NO:6, optionally further comprising one or more heterologous amino acid sequence; or (2) a nucleic acid encoding the polypeptide.

17. The composition of claim 16, wherein the polypeptide consists of the 15-55, 31-55, 20-92, or 20-55 segment of SEQ ID NO:1, the 10-51, 27-51, 16-88, or 16-51 segment of SEQ ID NO:3, or the 10-50, 26-50, 15-87, or 15-50 segment of SEQ ID NO:4, or the 10-51, 27-51, 16-88, or 16-51 segment of SEQ ID NO:5, or the 15-55, 31-55, 20-92, or 20-55 segment of SEQ ID NO:6.

18. The composition of claim 16, wherein the polypeptide consists of the 15-55, 31-55, 20-92, or 20-55 segment of SEQ ID NO:1, the 10-51, 27-51, 16-88, or 16-51 segment of SEQ ID NO:3, or the 10-50, 26-50, 15-87, or 15-50 segment of SEQ ID NO:4, or the 10-51, 27-51, 16-88, or 16-51 segment of SEQ ID NO:5, or the 15-55, 31-55, 20-92, or 20-55 segment of SEQ ID NO:6 and a poly-arginine or poly-histidine tag at the N-terminus.

19. The composition of claim 18, wherein the polypeptide consists of the 31-55 segment of SEQ ID NO:1, the 27-51 segment of SEQ ID NO:3, the 26-50 segment of SEQ ID NO:4, the 27-51 segment of SEQ ID NO:5, or the 31-55 segment of SEQ ID NO:6 and a poly-arginine or poly-histidine tag at the N-terminus.

20. A method for suppressing α-synuclein aggregation in a cell, comprising contacting the cell with an effective amount of (1) a polypeptide comprising the 31-55 segment of SEQ ID NO:1, the 27-51 segment of SEQ ID NO:3, or the 26-50 segment of SEQ ID NO:4, or the 27-51 segment of SEQ ID NO:5, or the 31-55 segment of SEQ ID NO:6, optionally further comprising one or more heterologous amino acid sequence; or (2) a nucleic acid encoding the polypeptide.

21. The method of claim 20, wherein the cell is a neuronal cell.

22. The method of claim 21, wherein the neuronal cell is in a human patient's body.

23. The method of claim 20, wherein the polypeptide consists of the 15-55, 31-55, 20-92, or 20-55 segment of SEQ ID NO:1, the 10-51, 27-51, 16-88, or 16-51 segment of SEQ ID NO:3, or the 10-50, 26-50, 15-87, or 15-50 segment of SEQ ID NO:4, or the 10-51, 27-51, 16-88, or 16-51 segment of SEQ ID NO:5, or the 15-55, 31-55, 20-92, or 20-55 segment of SEQ ID NO:6.

24. The method of claim 20, wherein the polypeptide consists of the 15-55, 31-55, 20-92, or 20-55 segment of SEQ ID NO:1, the 10-51, 27-51, 16-88, or 16-51 segment of SEQ ID NO:3, or the 10-50, 26-50, 15-87, or 15-50 segment of SEQ ID NO:4, or the 10-51, 27-51, 16-88, or 16-51 segment of SEQ ID NO:5, or the 15-55, 31-55, 20-92, or 20-55 segment of SEQ ID NO:6 and a poly-arginine or poly-histidine tag at the N-terminus.

25. The method of claim 24, wherein the polypeptide consists of the 31-55 segment of SEQ ID NO:1, the 27-51 segment of SEQ ID NO:3, the 26-50 segment of SEQ ID NO:4, the 27-51 segment of SEQ ID NO:5, or the 31-55 segment of SEQ ID NO:6 and a poly-arginine or poly-histidine tag at the N-terminus.

26. A method for treating a neurodegenerative disease in a human patient in need thereof, comprising administration to the patient an effective amount of:
(1) a polypeptide comprising the 31-55 segment of SEQ ID NO:1, the 27-51 segment of SEQ ID NO:3, or the 26-50 segment of SEQ ID NO:4, or the 27-51 segment of SEQ ID NO:5, or the 31-55 segment of SEQ ID NO:6, optionally further comprising one or more heterologous amino acid sequence; or
(2) a nucleic acid encoding the polypeptide.

27. The method of claim 26, wherein the administration comprises intravenous administration or transnasal administration.

28. The method of claim 26, wherein the polypeptide consists of the 15-55, 31-55, 20-92, or 20-55 segment of SEQ ID NO:1, the 10-51, 27-51, 16-88, or 16-51 segment of SEQ ID NO:3, or the 10-50, 26-50, 15-87, or 15-50 segment of SEQ ID NO:4, or the 10-51, 27-51, 16-88, or 16-51 segment of SEQ ID NO:5, or the 15-55, 31-55, 20-92, or 20-55 segment of SEQ ID NO:6.

29. The method of claim 26, wherein the polypeptide comprises the 15-55, 31-55, 20-92, or 20-55 segment of SEQ ID NO:1, the 10-51, 27-51, 16-88, or 16-51 segment of SEQ ID NO:3, or the 10-50, 26-50, 15-87, or 15-50 segment of SEQ ID NO:4, or the 10-51, 27-51, 16-88, or 16-51 segment of SEQ ID NO:5, or the 15-55, 31-55, 20-92, or 20-55 segment of SEQ ID NO:6 and a poly-arginine or poly-histidine tag at the N-terminus.

30. The method of claim 29, wherein the polypeptide consists of the 31-55 segment of SEQ ID NO:1, the 27-51 segment of SEQ ID NO:3, the 26-50 segment of SEQ ID NO:4, the 27-51 segment of SEQ ID NO:5, or the 31-55 segment of SEQ ID NO:6 and a poly-arginine or poly-histidine tag at the N-terminus.

31. The method of claim 26, wherein the neurodegenerative disease is Parkinson's disease.

32. The method of claim 31, wherein the Parkinson's disease is familial Parkinson's disease.

33. A kit for treating a neurodegenerative disease, comprising (1) a first container containing a polypeptide comprising the 31-55 segment of SEQ ID NO:1, the 27-51 segment of SEQ ID NO:3, or the 26-50 segment of SEQ ID NO:4, or the 27-51 segment of SEQ ID NO:5, or the 31-55 segment of SEQ ID NO:6, optionally further comprising one or more heterologous amino acid sequence, or a nucleic acid encoding the polypeptide; and (2) a second container containing a neuroprotective agent.

34. The kit of claim 33, wherein the polypeptide comprises the 15-55, 31-55, 20-92, or 20-55 segment of SEQ ID NO:1, the 10-51, 27-51, 16-88, or 16-51 segment of SEQ ID NO:3, or the 10-50, 26-50, 15-87, or 15-50 segment of SEQ ID NO:4, or the 10-51, 27-51, 16-88, or 16-51 segment of SEQ ID NO:5, or the 15-55, 31-55, 20-92, or 20-55 segment of SEQ ID NO:6.

35. The kit of claim 33, wherein the polypeptide consists of the 15-55, 31-55, 20-92, or 20-55 segment of SEQ ID NO:1, the 10-51, 27-51, 16-88, or 16-51 segment of SEQ ID NO:3, or the 10-50, 26-50, 15-87, or 15-50 segment of SEQ ID NO:4, or the 10-51, 27-51, 16-88, or 16-51 segment of SEQ ID NO:5, or the 15-55, 31-55, 20-92, or 20-55 segment of SEQ ID NO:6 and a poly-arginine or poly-histidine tag at the N-terminus.

36. The kit of claim 33, wherein the polypeptide consists of the 31-55 segment of SEQ ID NO:1, the 27-51 segment of SEQ ID NO:3, the 26-50 segment of SEQ ID NO:4, the 27-51 segment of SEQ ID NO:5, or the 31-55 segment of SEQ ID NO:6 and a poly-arginine or poly-histidine tag at the N-terminus.

37. The kit of claim 33, further comprising an instruction manual.
